SEQUENCE LISTING (6 sequences)

SEQ ID NO: 1 - DNA, 1618 bp, Unknown
  Bacteriophage lysogen #33 in Acinetobacter baumannii
  misc_feature (1)..(1618): terSL region deleted terS (full), terL until 150 bp before 3′ stop codon
atggctgcacttaaagaacaggtaaaaatatttattgttcaagcgcttgcctgcatggatacccctcaacaggtagctaatgctgtcaagcaagaatttaacattgagattgatcgaaaacaggtacaactttatgacccgacaaaagcggcaggaaagaatttaagtaagaaatataaagacctttttcataaaacccgagaggactttaaaaagaatgtttatgacatccctttagctaataaagcctatcggcttaaagaacttcagaagatctatgaagactggaagaacaacaggcttatgaagcaaggggttattaaacaggttagggaagaaatgcagggttatgacctcatgcttttaaatcttgagttaaagcaacttgagattgaaaagttaagagagggtgaaggtgatgaagatccaacaccagtcaaggtaactattcaagttgtagatgcgagtaaaaaagatgccgaacatcaatccgacactgaatgtacctcaggctaattttttgcagatggaaaagaagttccgcgcatttgtcgctggctttggatcgggaaagacttgggttggatgctccagtttatgcaacaaagcttgggaattcccaaaagtacctttgggttattttgctccaacttacccgcagattcgcgacattttctttccaactattgaagaggttgctttcgattgggggcttaaaactaaggtttatgaaaccaataaagaggtggatatctattatggtcggcaatatcgaactacaatcatttgccggtctatggagaaaccagcaaccattgtaggttttaaaattggccacgccttgattgatgagcttgatgttatggccaaggtcaaagctcaacaggcttggcgtaagatcatcgctcgtatgcgttataagcaagctggtttgctcaacggtattgatgtggccacaacaccagaaggttttaagtttacatacgagcaatttgttaaagaggcaaataaatcagaggctaagcgtaagctctatggaatgattcaagcttcaacttatgacaatgaagctaatcttccagatgactacatatcatcactttatgagtcttatccgccgcaattaatttcagcttatttaagagggcagtttgtcaatttaaccagtggtgctgtttaccccgactttgatcgagttctaaaccacacggatgaagaaattaagaaaggtgagcctttactcattggtatggattttaacgtgcttaaaatggctgctgtggtttatgtcattagagaagggaagccaagagctttagatgaactggttggcgtgagagatacaccgacgatgtgtcaactgattaatgagcgctttccagatcacgatattactgtgattccagatgcttcaggtcaggcaacatcatcaaagaacttcagtgaatctgatcatgcaatcttaaagaaaaatggattcaaagttgaagttaatggtgtgaatcccggtattaaagatcgtatcactgcagttaatgcacaaattctgaatgctgagggtgaacgacacttaaaagtgaacacaaacaagtgtcctaactttacggctactttagaa

SEQ ID NO: 2 - DNA, 1842 bp, Unknown
  Bacteriophage lysogen #49 in Acinetobacter baumannii
  misc_feature (1)..(1842): terSL region deleted 58 bp upstream of terS, terS (full), and terL until 92 bp before 3′ stop codon
gcctaatggctcttttttttgcccattttgttatacgtagttatacgatgaggaagttatggcgacactaaaagagcctgtgaaaatctttatagttcagtctcttgcttgtcgtgatacacctcaagaagtggctgaactcgtaaaacaagaatttggcgttgatatagatcgtgttcaagttgcaacatatgaccctacaaaagttgctggtaagaacttaagcaaaaagtatgtcgaactatttgaaaaaaccagagatgagtttgataaaggcttaattgatattcctattgctaataagtactaccgattgaagcaataccaaagacaacttgagaagactagaaacgttaaaacagccttaaaaattcttgagcaagccgctaaagacattggtggtcaatttactaatcgccaagaaattacaggcaaagacggcggaccagtccaaacagttaattctgaaattcaagttccaatggaagattacttaaaagcacggagggaagtcttagatgagtactgatgcggctcgggataaagccatccggatcgaggcgcaagaagatttatatttcttcacaaggtacatgtttaaggagcgccgtggttataaatggatgcaaaattggcaccacttagaaatctgcgaagctttaatgaaagtttatcgcggagagataaagcggttaattattaacgttccaccacgatattctaaaactgaaattgctgtaattaatttcatggcttggtgttttggtaagaatccagactgtgagtttattcatatcagttactcggcaatgcttgccgcaaataatgccttccagattcgaacactcgtacaagaggaggcgtataaaaaggtctttcctgatcttacattgcgtgatgatagtaaggctaaagacttctggagaacttctcaaggcggtgtctgctatgcgactggtacaggcggtacgattactggttttggcgcaggtaaacttcgtgatgggtttggtggatgcatcattatcgatgacccacacaaagcgcatgaagcttcttctaaaacaattcgagaaggggtaattgattggttccaaaacacccttgagtcgcgtactaactcaccagatacgccgatcattgtgattatgcagcgacttcatgaagatgatttggctggttggttgttaggcgatagaaaagatggcgttcctgtagctggtggtaacggtgaggtatgggaacatctttgtctttctgctattcaggaagacggatccgcactatggccagcaaaacacaatatccaaaagttaaggcaaatggagcaagctgcgccgtatgtatttgcagggcaataccgacaaatgccatcaccgccagcaggcggtttttttaagcctgacaatattcaaattgttgatgctttgcctgcggatgttttgaaacaagtgagggcttgggacttcggagcgaccgaaaacgaaggcgactttactgtaggtgtaagagaagctctaggtgcagatggttttacttacattgtcgatgttactagaggacagcttggtccagacaatgtgaataagcgcttagaacaaaccgcaaaaatagatgggaaaaaagtttctgtgcgtctaccacaagatcccggtcaagctggtaaatcgcaagctagttcatttgtgaagcttcttgcgggttatagcgtgatagctaagccaatttcaggtgacaagcttacacgtgcacaaccatttgcggcccaagttaacgtaggaaatgtacgaatgctcaaaggtgaatggaataaggattttattgatgagcttcgtcattttc

SEQ ID NO: 3 - DNA, 2679 bp, Unknown
  Bacteriophage lysogen #147 in Acinetobacter baumannii
  misc_feature (1)..(2679): terSL region deleted128 bp upstream of terS, terS (full), terL (full)
ggatgtcaaaacttccaagcccttcgccgttggacaccgccccccatcgcacgcacaaaaaaaattccctctcagaaaaagttaaagcaaaaagttaaaatcaagttaaaggtagagcaatggcattaacagagaaaatggaaaaatttgctcttgccattgttgacggcaagacaaataaagaagcagcaatttcagcaggttatgcggaaaaaactgcatccgccgcaggtgctaggttagcaaaagatcctgaaattattgtctatattgaaatgttaaaggcccaaaaagaagggcgctctttaacatctaattcaccaaaagttaaacctaaagatacacccgaaaatagtggtgaagatgaaaaccctattgaggaatttcaatttgaaggtgatgatcctttagattttttaattaaggtcatgaacttcaatggcaacaagctgccacttagaatgcaagcagcaattgcagcattgccttataaacacgggaaggttgcggaaaaaggcaaaaaagaaactaaacaagacaaggcaaaggaagcgaccagaacaggaaaatacgccacattggacaatcagttgccaagctaactattaagaggaactttgcatgcaaagtctagaatacgaaaccgtaagtggtgaaactatcactattcaaaatatcaaagatggcccttgctgtcatgactctattgaaaagttgccagcaacagaaaggttggtgaagattacttatcaatgtcgcaagtgcttttccagattttccgaagaagattatcaattgattgttaatcaataaaaggttttgtatggatccgtaggcgatacggtgcgttggaggaaggaggaccacaactgccaacgtaataacccgctagcagtgggcgaaacagcgtagttaaagcaggggttcgcaacctgtcatacaaatttattccgccttcgggcggttttttcatggaccatttaaatgactgcaaaactaccagactggactacagcttgcccagactgggcgacccgtattgtttctaaacaatcgttaatgccgtgtaagccattattccccaaagtggctgacgtagcggagcgtatctttaaagagttaattcttgttgatgtgatgggtagccctaagatgggtgatgtcacattggaatgggtgatcgagtttgttcgtgcaatctttggcgcatatgatccaagcacaaagcgcagattaattcgtgaattctttcttttgatttcgaagaagaatactaaatctacgattgccgccggcattatgcttactgcattaattcttaatgatcgacaatctgccgaactaattattcttgcgcctactaaagaagttgctgataactcatttaatccaatccgggatttcatacgcgcagatgaagaattaagtgaaagatttaatgtatctgagcacacaaaaacagttacgcatctaggtaccggagcaacacttaaagttattgcagcagaatctaacgctgcagctggtaagaaagcttcaatcattttgatagatgaggtctggctattcgggaaacgtgccaacgctgaatcaatgttccgtgaagcaaagggtggtttagcatctcgtccagaaggttgtgtgatttatctgtctaccatgtcggatgaagtgccatgtggagtatttaagcagcttttagattatgccagagatgtacgtgacggaattaaagttgataaaagttttctaccacttatttatgaattccctaagcatcttgtagaagcaggcgaacatttaaaacctgaaaatttctacatcacaaacccaaacttgggtgcttcggttgatcttgaatatctgatttcggaatttaacaaagttaaagatgctagtgaagaatctcttagagacttcttggccaaacacttaaacattgaaatcggcatgaaccttcgtgctaaccggtgggcgggtgcagagtattggaatgctcaagctaaagatatccaaatcgaccaactaattgagctatccgatgtcattactttgggtattgatggcggtggtctcgacgacttacttggcttcgctgctttaggtcgtttaacagaagatcctcgtatctggtggctatggaatcatgcatgggcaaataagattgctttagagcgcagaaaagagaatgtgcctaagtatgaagacttcaagtctgagggttctctaactgttgttgaccgaataggcgatgacattgaccaactcgcagcaattgctaagaaggtttatgacagtggaaagcttaataagatcggactagatccattgggcttaggcggtcttttagatggcttacttgaggcaggaattccagaggaaagcatgtttgctgtgccacaaggctacaaactcatgtcctacatccttactactgagcgcaaattggcagaaggcaatctgtaccatgctggacaacagctaatgacttgggcggcaggtaatgcccgtgtcgtgatggtcggcaatggtatgcgaataaccaagcaagaatcaggtgttgggaagattgacccattgattgccacatttaacgcagttgctttgatgtcaagcaatcctgagcctgccaatcgcgttgatattgacgaatacttagaggatgtcgtgatagcatga

SEQ ID NO: 4 - DNA, 9247 bp, Artificial Sequence
  Synthesized plasmid pZX058 with Abi 33 terSL region
gaagatcctttgatcttttctacggggtctgacgctcagtggaacgaaaactcacgttaagggattttggtcatgagattatcctcgtcaggtggcacttttcggggaaatgtgcgcggaacccctatttgtttatttttctaaatacattcaaatatgtatccgctcatgagacaataaccctgataaatgcttcaataatattgaaaaaggaagagtcgatcgatgaagtttggaaatatttgtttttcgtatcaaccaccaggtgaaactcataagcaagtaatggatcgctttgttcggcttggtatcgcctcagaagaggtagggtttgatacatattggaccttagaacatcattttacagagtttggtcttacgggaaatttatttgttgctgcggctaacctgttaggaagaactaaaacattaaatgttggcactatgggggttgttattccgacagcacacccagttcgacagttagaagacgttttattattagatcaaatgtcgaaaggtcgttttaattttggaaccgttcgagggctataccataaagattttcgagtatttggtgttgatatggaagagtctcgagcaattactcaaaatttctaccagatgataatggaaagcttacagacaggaaccattagctctgatagtgattacattcaatttcctaaggttgatgtatatcccaaagtgtactcaaaaaatgtaccaacccgtatgactgctgagtccgcaagtacgacagaatggctagcaatacaagggctaccaatggttcttagttggattattggtactaatgaaaaaaaagcacagatggaactctataatgaaattgcgacagaatatggtcatgatatatctaaaatagatcattgtatgacttatatttgttctgttgatgatgatgcacaaaaggcgcaagatgtttgtcgggagtttctgaaaaattggtatgactcatatgtaaatgcgaccaatatctttaatgatagcgatcaaactcgtggttatgattatcataaaggtcaatggcgtgattttgttttacaaggacatacaaacaccaatcgacgtgttgattatagcaatggtattaaccccgtaggcactcctgagcagtgtattgaaatcattcaacgtgatattgatgcaacgggtattacaaacattacatgcggatttgaagctaatggaactgaagatgaaataattgcttccatgcgacgctttatgacacaagtcgctcctttcttaaaagaacctaaataaattacttatttgatactagagataataaggaacaagttatgaaatttggattattttttctaaactttcagaaagatggaataacatctgaagaaacgttggataatatggtaaagactgtcacgttaattgattcaactaaatatcattttaatactgcctttgttaatgaacatcacttttcaaaaaatggtattgttggagcacctattaccgcagctggttttttattagggttaacaaataaattacatattggttcattaaatcaagtaattaccacccatcaccctgtacgtgtagcagaagaagccagtttattagatcaaatgtcagagggacgcttcattcttggttttagtgactgcgaaagtgatttcgaaatggaattttttagacgtcatatctcatcaaggcaacaacaatttgaagcatgctatgaaataattaatgacgcattaactacaggttattgccatccccaaaacgacttttatgattttccaaaggtttcaattaatccacactgttacagtgagaatggacctaagcaatatgtatccgctacatcaaaagaagtcgtcatgtgggcagcgaaaaaggcactgcctttaacgtttaagtgggaggataatttagaaaccaaagaacgctatgcaattctatataataaaacagcacaacaatatggtattgatatttcggatgttgatcatcaattaactgtaattgcgaacttaaatgctgatagaagtacggctcaagaagaagtgagagaatacttaaaagactatatcactgaaacttaccctcaaatggacagagatgaaaaaattaactgcattattgaagagaatgcagttgggtctcatgatgactattatgaatcgacaaaattagcagtggaaaaaacagggtctaaaaatattttattatcctttgaatcaatgtccgatattaaagatgtaaaagatattattgatatgttgaaccaaaaaatcgaaatgaatttaccataaggatcctaaaattaaaggcaatttctatattagattgcctttttggcgcgcctattctaatgcataataaatactgataacatcttatattttgtattatattttgtattatcgttgacatgtataattttgatatcaaaaactgattttccctctattattttcgagatttattttcttaattctctttaacaaactagaaatattgtatatacaaaaaattataaataatagatgaatagtttaattataggtgttcatcaatcgaaaaagcaacgtatcttatttaaagtgcgttgcttttttctcatttataaggttaaataattctcatatatcaagcaaagtgacagagctcggtactctcggcttgaacgaattggcggccgccctgcaggatagctgacgaccttaaggataaatttctggtaaggaggacacgtatggaagtgggcaagttggggaagccgtatccgttgctgaatctggcatatgtgggagtataagacgcgcagcgtcgcatcaggcatttttttctgcgccaatgccggtagatagcaatccggcccgaggggcacaaaaaacccctcaagacccgtttagaggccccaaggggttatgctacgatactcaggcgccgggggcggtgtccggcggcccccagaggaactgcgccagttcctccggatcggtgaagccggagagatccagcggggtctcctcgaacacctcgaagtcgtgcaggaaggtgaaggcgagcagttcgcgggcgaagtcctcggtccgcttccactgcgccccgtcgagcagcgcggccaggatctcgcggtcgccccggaaggcgttgagatgcagttgcaccaggctgtagcgggggtctcccgcatagacgtcggtgaagtcgacgatcccggtgacctcggtcgcggccaggtccacgaagatgttggtcccgtgcaggtcgccgtggacgaaccggggttcgcggccggccagcagcgtgtccacgtccggcagccagtcctccaggcggtccagcagccggggcgagaggtagccccacccgcggtggtcctcgacggtcgccgcgcggcgttcccgcagcagttccgggaagacctcggaatggggggtgagcacggtgttcccggtcagcggcaccctgtgcagccggccgagcacccggccgagttcgcgggccagggcgagcagcgcgttccggtcggtcgtgccgtccatcgcggaccgccaggtggtgccggtcacccggctcatcaccaggtagggccacggccaggctccggtgccgggccgcagctcgccgcggacgaggaggcggggcaccggcaccggggcgtccgccaggaccgcgtacgcctccgactccgacgcgaggctctccggaccgcaccagtgctcgccgaacagcttgatcaccgggccgggctcgccgaccagtacggggttggtgctctcgccgggcacccgcagcaccggcggcaccggcagcccgagctcctccagggctcggcgggccagcggctcccagaattcctggtcgttccgcaggctcgcgtaggaatcatccgaatcaatacggtcgagaagtaacagggattcttgtgtcatatgaatcttactcctttgttaaattatttttgtttaagcattttgaatttgtatgcatcttagaacaatctcaaagttccaagctcttgcaagtctagatattgagtcaaaaaaaattttcaactcatactcttcctttttcaatattattgaagcatttatcagggttattgtctcatgagcgcacatttccccgaaaagtgccacctgacgtctaagaaaccagcggccgctcacactggctcaccttcgggtgggcctttctgcgtttatatactagagagagaatataaaaagccagattattaatccggcttttttattatttgggtgcctaatgagtgagctatggagcagatcagaccatttccaccgactgattttattgatcaagcagatgaagaagaagcaataagactaacaccagcaccagatctaaaaaaatgggttgttgctaattacttaactattggtggacctctttataatcccgatcatgatcacatagctgagctgcttcacgataatgaagaatttttagcatgtgcttgggcctcttctgcatataaaagcaagcaagctatggtgttaggtcagtgcgaaaaagtcatgttcaatgttggtggatggcgtaaggccagacaagagcaacagatgcgagactggttcggctttgtgccaacatacttgatcaccattgatgctacattttgcgacaaagcaaatgatcgtgagttttgtgctttgcttgagcatgaactctaccatataggcgtagaacgtgatgaagacggtgaaatgatctttagtagctcaacaggtttacctaaacattatttagctggtcacgatgtcgaagagtttgttggtgtaaccaaacggtggggggcgagtcaaagcgttaaacgtatcgttgaagctgcaaagaatccgccgtttgtttcgaaacttgatatttcaaaatgctgcggaaactgcgtaatcaactgagccgaatggctcttttttttgccttctttgctagacgtagctagacaaaggtgggggtatggctgcacttaaagaacaggtaaaaatatttattgttcaagcgcttgcctgcatggatacccctcaacaggtagctaatgctgtcaagcaagaatttaacattgagattgatcgaaaacaggtacaactttatgacccgacaaaagcggcaggaaagaatttaagtaagaaatataaagacctttttcataaaacccgagaggactttaaaaagaatgtttatgacatccctttagctaataaagcctatcggcttaaagaacttcagaagatctatgaagactggaagaacaacaggcttatgaagcaaggggttattaaacaggttagggaagaaatgcagggttatgacctcatgcttttaaatcttgagttaaagcaacttgagattgaaaagttaagagagggtgaaggtgatgaagatccaacaccagtcaaggtaactattcaagttgtagatgcgagtaaaaaagatgccgaacatcaatccgacactgaatgtacctcaggctaattttttgcagatggaaaagaagttccgcgcatttgtcgctggctttggatcgggaaagacttgggttggatgctccagtttatgcaacaaagcttgggaattcccaaaagtacctttgggttattttgctccaacttacccgcagattcgcgacattttctttccaactattgaagaggttgctttcgattgggggcttaaaactaaggtttatgaaaccaataaagaggtggatatctattatggtcggcaatatcgaactacaatcatttgccggtctatggagaaaccagcaaccattgtaggttttaaaattggccacgccttgattgatgagcttgatgttatggccaaggtcaaagctcaacaggcttggcgtaagatcatcgctcgtatgcgttataagcaagctggtttgctcaacggtattgatgtggccacaacaccagaaggttttaagtttacatacgagcaatttgttaaagaggcaaataaatcagaggctaagcgtaagctctatggaatgattcaagcttcaacttatgacaatgaagctaatcttccagatgactacatatcatcactttatgagtcttatccgccgcaattaatttcagcttatttaagagggcagtttgtcaatttaaccagtggtgctgtttaccccgactttgatcgagttctaaaccacacggatgaagaaattaagaaaggtgagcctttactcattggtatggattttaacgtgcttaaaatggctgctgtggtttatgtcattagagaagggaagccaagagctttagatgaactggttggcgtgagagatacaccgacgatgtgtcaactgattaatgagcgctttccagatcacgatattactgtgattccagatgcttcaggtcaggcaacatcatcaaagaacttcagtgaatctgatcatgcaatcttaaagaaaaatggattcaaagttgaagttaatggtgtgaatcccggtattaaagatcgtatcactgcagttaatgcacaaattctgaatgctgagggtgaacgacacttaaaagtgaacacaaacaagtgtcctaactttacggctactttagaacagcaagtctatgatgattttggaatgccagataaaagcgctggtttggaccacgttggggacgctggtggatatccaatagctaagagattcccagtcatcattcagaaaatatttaaacggcgcgcaatcgctggtttttctcgttaatcaatgcaccttctcaggtgcttttttattggtgtttttatggcagttactgataaacatccgcagtatattgctgcacaaaaaagctgggagattatgcgggacgccgttgctggtgaagagcagatcaaacaggcacaaacaaagtacctagctaaatcggccggaatgattgaggctgaaaagcaaggtgatacgactggagagatttataaggcctatctaagtcgagctcagtatccgctatgggttcaggacgcattacgcacgactcacattaattgcgttgcgctcacgatcgtagaaatatctatgattatcttgaagaacgcaaccctatagcagctattgaaattgatgatttaattgaagaaaagacagatttacttgttgataatcgactgatggggcgcacaggcagacagaaagatactagggagttagtgatacatccgcattatgtggttgtatatgacatcactgatataatacggatactcagagtgctacacacatcgcaggagtggtcatgacttactcatgtactttggattatttagtgttataaaatcctgatttataaattttttttgttaaaaaagataaaagccccttgcaattgcttggggctttaccgtaatttatggggtacagatcttcgatactgacatatcggcaatcgaaagcattaaggtttgacgaccgctaatgatttcaccacaggggcttaatgtacctgtcttaaattctaaggttttaactcgctttgtcaagcatagaccccaaaaatttagccaatgtctgtaactcaatctgtccatgtgtgggtgatgaggtacagtgacgctagcacacatcggaaaaacgctattactaggggaactgaacagagtagcggacgcaatgagtagtcatttaattggcggttatgagcgtgttcaggcggtgctatcaatcgtaatcataacagtggcagcttgatacagtgatgtcatccctgatgcgaaagcgaccgaccgacggtacatcgaatgggaatactttagggtgatttttaagaatcgctctagggtgagtatttcccattcagctctgctccctccctctggtactttaatcaaaagcactactaaacatatgtttttaaataaaaaatattgatatagagataatattagtaagaataattaaacaattgaatatagataaatcattgttaaataaagattaattattaaaatgaatgtatacttatatataaatcaatgatttaaaatatttgataaagaaaacttttcaaaaaaaatataattgagattgtgtcatttcggtcaattcttaatatgttccacgcaagttttagctatggtgctaaacagaaatttgctgaaaaagaacttttcactgaactggttaaaatgtaagcagcctgagagccgccaaaaattttaaaaacaaaccgccttaatcatcttcaaaaaatacctctaaaacctcaccatttgcgttttaagacccatatttcatcctgcccttatgttcccatgctgatagctataaagtgtctgtaatcgcttcctatgacgttctaggctgttgataacttttggaacaacgcaaaatgttaaaatcctgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 5 - DNA, 9463 bp, Artificial Sequence
  Synthesized plasmid pZX065 with Abi 49 terSL region
gaagatcctttgatcttttctacggggtctgacgctcagtggaacgaaaactcacgttaagggattttggtcatgagattatcctcgtcaggtggcacttttcggggaaatgtgcgcggaacccctatttgtttatttttctaaatacattcaaatatgtatccgctcatgagacaataaccctgataaatgcttcaataatattgaaaaaggaagagtcgatcgatgaagtttggaaatatttgtttttcgtatcaaccaccaggtgaaactcataagcaagtaatggatcgctttgttcggcttggtatcgcctcagaagaggtagggtttgatacatattggaccttagaacatcattttacagagtttggtcttacgggaaatttatttgttgctgcggctaacctgttaggaagaactaaaacattaaatgttggcactatgggggttgttattccgacagcacacccagttcgacagttagaagacgttttattattagatcaaatgtcgaaaggtcgttttaattttggaaccgttcgagggctataccataaagattttcgagtatttggtgttgatatggaagagtctcgagcaattactcaaaatttctaccagatgataatggaaagcttacagacaggaaccattagctctgatagtgattacattcaatttcctaaggttgatgtatatcccaaagtgtactcaaaaaatgtaccaacccgtatgactgctgagtccgcaagtacgacagaatggctagcaatacaagggctaccaatggttcttagttggattattggtactaatgaaaaaaaagcacagatggaactctataatgaaattgcgacagaatatggtcatgatatatctaaaatagatcattgtatgacttatatttgttctgttgatgatgatgcacaaaaggcgcaagatgtttgtcgggagtttctgaaaaattggtatgactcatatgtaaatgcgaccaatatctttaatgatagcgatcaaactcgtggttatgattatcataaaggtcaatggcgtgattttgttttacaaggacatacaaacaccaatcgacgtgttgattatagcaatggtattaaccccgtaggcactcctgagcagtgtattgaaatcattcaacgtgatattgatgcaacgggtattacaaacattacatgcggatttgaagctaatggaactgaagatgaaataattgcttccatgcgacgctttatgacacaagtcgctcctttcttaaaagaacctaaataaattacttatttgatactagagataataaggaacaagttatgaaatttggattattttttctaaactttcagaaagatggaataacatctgaagaaacgttggataatatggtaaagactgtcacgttaattgattcaactaaatatcattttaatactgcctttgttaatgaacatcacttttcaaaaaatggtattgttggagcacctattaccgcagctggttttttattagggttaacaaataaattacatattggttcattaaatcaagtaattaccacccatcaccctgtacgtgtagcagaagaagccagtttattagatcaaatgtcagagggacgcttcattcttggttttagtgactgcgaaagtgatttcgaaatggaattttttagacgtcatatctcatcaaggcaacaacaatttgaagcatgctatgaaataattaatgacgcattaactacaggttattgccatccccaaaacgacttttatgattttccaaaggtttcaattaatccacactgttacagtgagaatggacctaagcaatatgtatccgctacatcaaaagaagtcgtcatgtgggcagcgaaaaaggcactgcctttaacgtttaagtgggaggataatttagaaaccaaagaacgctatgcaattctatataataaaacagcacaacaatatggtattgatatttcggatgttgatcatcaattaactgtaattgcgaacttaaatgctgatagaagtacggctcaagaagaagtgagagaatacttaaaagactatatcactgaaacttaccctcaaatggacagagatgaaaaaattaactgcattattgaagagaatgcagttgggtctcatgatgactattatgaatcgacaaaattagcagtggaaaaaacagggtctaaaaatattttattatcctttgaatcaatgtccgatattaaagatgtaaaagatattattgatatgttgaaccaaaaaatcgaaatgaatttaccataaggatcctaaaattaaaggcaatttctatattagattgcctttttggcgcgcctattctaatgcataataaatactgataacatcttatattttgtattatattttgtattatcgttgacatgtataattttgatatcaaaaactgattttccctctattattttcgagatttattttcttaattctctttaacaaactagaaatattgtatatacaaaaaattataaataatagatgaatagtttaattataggtgttcatcaatcgaaaaagcaacgtatcttatttaaagtgcgttgcttttttctcatttataaggttaaataattctcatatatcaagcaaagtgacagagctcggtactctcggcttgaacgaattggcggccgccctgcagatgaatcagatcagaccatttcctccaactgattttatggatcaggccgaagaagaggaagcaattcgtttaataccggctccagacctaaagaaatgggttgtggctaattacttaactattgggggtcctatttataatccagatcatgatcatattgctgagctgcttcatgataatgacgagtttttagcattcgcgtgggcctcttctgcatataaaagcaagcaagctatggtgttaggccagtgcgaaaaagtcatgttcaatgttggtggctggcgtaaagctcgacaagagcaacagatgcgtgattggtttggttttgtacctacttatttaataactgtcgacgcttctttctgtgagcgtgcaaacgatacagagttctgttacttacttgaacatgagctttaccacattggagtggtgagagacgaggacggagaaattgtttatagcgatagttctggtcttcctaagcactatcttgctggtcatgacgttgaagagtttattggcgtagttaaacgttatggaccaagcaaaaatgttaagcgacttattgaagtcgcaaaaaatccgccgtttgtttcgaatcttgatatttcaaaatgctgcggaaactgtgtaatcaattgagcctaatggctcttttttttgcccattttgttatacgtagttatacgatgaggaagttatggcgacactaaaagagcctgtgaaaatctttatagttcagtctcttgcttgtcgtgatacacctcaagaagtggctgaactcgtaaaacaagaatttggcgttgatatagatcgtgttcaagttgcaacatatgaccctacaaaagttgctggtaagaacttaagcaaaaagtatgtcgaactatttgaaaaaaccagagatgagtttgataaaggcttaattgatattcctattgctaataagtactaccgattgaagcaataccaaagacaacttgagaagactagaaacgttaaaacagccttaaaaattcttgagcaagccgctaaagacattggtggtcaatttactaatcgccaagaaattacaggcaaagacggcggaccagtccaaacagttaattctgaaattcaagttccaatggaagattacttaaaagcacggagggaagtcttagatgagtactgatgcggctcgggataaagccatccggatcgaggcgcaagaagatttatatttcttcacaaggtacatgtttaaggagcgccgtggttataaatggatgcaaaattggcaccacttagaaatctgcgaagctttaatgaaagtttatcgcggagagataaagcggttaattattaacgttccaccacgatattctaaaactgaaattgctgtaattaatttcatggcttggtgttttggtaagaatccagactgtgagtttattcatatcagttactcggcaatgcttgccgcaaataatgccttccagattcgaacactcgtacaagaggaggcgtataaaaaggtctttcctgatcttacattgcgtgatgatagtaaggctaaagacttctggagaacttctcaaggcggtgtctgctatgcgactggtacaggcggtacgattactggttttggcgcaggtaaacttcgtgatgggtttggtggatgcatcattatcgatgacccacacaaagcgcatgaagcttcttctaaaacaattcgagaaggggtaattgattggttccaaaacacccttgagtcgcgtactaactcaccagatacgccgatcattgtgattatgcagcgacttcatgaagatgatttggctggttggttgttaggcgatagaaaagatggcgttcctgtagctggtggtaacggtgaggtatgggaacatctttgtctttctgctattcaggaagacggatccgcactatggccagcaaaacacaatatccaaaagttaaggcaaatggagcaagctgcgccgtatgtatttgcagggcaataccgacaaatgccatcaccgccagcaggcggtttttttaagcctgacaatattcaaattgttgatgctttgcctgcggatgttttgaaacaagtgagggcttgggacttcggagcgaccgaaaacgaaggcgactttactgtaggtgtaagagaagctctaggtgcagatggttttacttacattgtcgatgttactagaggacagcttggtccagacaatgtgaataagcgcttagaacaaaccgcaaaaatagatgggaaaaaagtttctgtgcgtctaccacaagatcccggtcaagctggtaaatcgcaagctagttcatttgtgaagcttcttgcgggttatagcgtgatagctaagccaatttcaggtgacaagcttacacgtgcacaaccatttgcggcccaagttaacgtaggaaatgtacgaatgctcaaaggtgaatggaataaggattttattgatgagcttcgtcattttcctaatggcacacatgacgaccaagtggatgcagcttcagatgcgtttaatgaattacatgaaggttttgaagccttctttgctgatatgggatttgcacgatgagtgatgtaacttttcaacatcctgaatatgttaaaaacttgccatactggcaaaaacttgatgatgtttgtgaaggtgaagatgcagttaaggctaaaggtgaaaaatatttgccgatgccaaatgcacatgataaatcacctgcaaataaaagcgcttatgaggcttatcttactcgtgcagtcttttatgaagtaacagggactacatcaaatagtttagttggagcagcttttgcaacagatccaagttttaaatttcctcccgaccttgctcatttagaacgtaatgcgaatggagccggtttaagtacttatcaattggctcaaaatggaattcgccacttattgaagcattatcgttgcggatagctgacgaccttaaggataaatttctggtaaggaggacacgtatggaagtgggcaagttggggaagccgtatccgttgctgaatctggcatatgtgggagtataagacgcgcagcgtcgcatcaggcatttttttctgcgccaatccggtagatagcaatccggcccgaggggcacaaaaaacccctcaagacccgtttagaggccccaaggggttatgctacgatactcaggcgccgggggcggtgtccggcggcccccagaggaactgcgccagttcctccggatcggtgaagccggagagatccagcggggtctcctcgaacacctcgaagtcgtgcaggaaggtgaaggcgagcagttcgcgggcgaagtcctcggtccgcttccactgcgccccgtcgagcagcgcggccaggatctcgcggtcgccccggaaggcgttgagatgcagttgcaccaggctgtagcgggggtctcccgcatagacgtcggtgaagtcgacgatcccggtgacctcggtcgcggccaggtccacgaagatgttggtcccgtgcaggtcgccgtggacgaaccggggttcgcggccggccagcagcgtgtccacgtccggcagccagtcctccaggcggtccagcagccggggcgagaggtagccccacccgcggtggtcctcgacggtcgccgcgcggcgttcccgcagcagttccgggaagacctcggaatggggggtgagcacggtgttcccggtcagcggcaccctgtgcagccggccgagcacccggccgagttcgcgggccagggcgagcagcgcgttccggtcggtcgtgccgtccatcgcggaccgccaggtggtgccggtcacccggctcatcaccaggtagggccacggccaggctccggtgccgggccgcagctcgccgcggacgaggaggcggggcaccggcaccggggcgtccgccaggaccgcgtacgcctccgactccgacgcgaggctctccggaccgcaccagtgctcgccgaacagcttgatcaccgggccgggctcgccgaccagtacggggttggtgctctcgccgggcacccgcagcaccggcggcaccggcagcccgagctcctccagggctcggcgggccagcggctcccagaattcctggtcgttccgcaggctcgcgtaggaatcatccgaatcaatacggtcgagaagtaacagggattcttgtgtcatatgaatcttactcctttgttaaattatttttgtttaagcattttgaatttgtatgcatcttagaacaatctcaaagttccaagctcttgcaagtctagatattgagtcaaaaaaaattttcaactcatactcttcctttttcaatattattgaagcatttatcagggttattgtctcatgagcgcacatttccccgaaaagtgccacctgacgtctaagaaaccagcggccgctcacactggctcaccttcgggtgggcctttctgcgtttatatactagagagagaatataaaaagccagattattaatccggcttttttattatttgggtgcctaatgagtgagctaactcacattaattgcgttgcgctcacgatcgtagaaatatctatgattatcttgaagaacgcaaccctatagcagctattgaaattgatgatttaattgaagaaaagacagatttacttgttgataatcgactgatggggcgcacaggcagacagaaagatactagggagttagtgatacatccgcattatgtggttgtatatgacatcactgatataatacggatactcagagtgctacacacatcgcaggagtggtcatgacttactcatgtactttggattatttagtgttataaaatcctgatttataaattttttttgttaaaaaagataaaagccccttgcaattgcttggggctttaccgtaatttatggggtacagatcttcgatactgacatatcggcaatcgaaagcattaaggtttgacgaccgctaatgatttcaccacaggggcttaatgtacctgtcttaaattctaaggttttaactcgctttgtcaagcatagaccccaaaaatttagccaatgtctgtaactcaatctgtccatgtgtgggtgatgaggtacagtgacgctagcacacatcggaaaaacgctattactaggggaactgaacagagtagcggacgcaatgagtagtcatttaattggcggttatgagcgtgttcaggcggtgctatcaatcgtaatcataacagtggcagcttgatacagtgatgtcatccctgatgcgaaagcgaccgaccgacggtacatcgaatgggaatactttagggtgatttttaagaatcgctctagggtgagtatttcccattcagctctgctccctccctctggtactttaatcaaaagcactactaaacatatgtttttaaataaaaaatattgatatagagataatattagtaagaataattaaacaattgaatatagataaatcattgttaaataaagattaattattaaaatgaatgtatacttatatataaatcaatgatttaaaatatttgataaagaaaacttttcaaaaaaaatataattgagattgtgtcatttcggtcaattcttaatatgttccacgcaagttttagctatggtgctaaacagaaatttgctgaaaaagaacttttcactgaactggttaaaatgtaagcagcctgagagccgccaaaaattttaaaaacaaaccgccttaatcatcttcaaaaaatacctctaaaacctcaccatttgcgttttaagacccatatttcatcctgcccttatgttcccatgctgatagctataaagtgtctgtaatcgcttcctatgacgttctaggctgttgataacttttggaacaacgcaaaatgttaaaatcctgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 6 - DNA, 9768 bp, Artificial Sequence
  Synthesized plasmid pZX066 with Abi 147 terSL region
gaagatcctttgatcttttctacggggtctgacgctcagtggaacgaaaactcacgttaagggattttggtcatgagattatcctcgtcaggtggcacttttcggggaaatgtgcgcggaacccctatttgtttatttttctaaatacattcaaatatgtatccgctcatgagacaataaccctgataaatgcttcaataatattgaaaaaggaagagtcgatcgatgaagtttggaaatatttgtttttcgtatcaaccaccaggtgaaactcataagcaagtaatggatcgctttgttcggcttggtatcgcctcagaagaggtagggtttgatacatattggaccttagaacatcattttacagagtttggtcttacgggaaatttatttgttgctgcggctaacctgttaggaagaactaaaacattaaatgttggcactatgggggttgttattccgacagcacacccagttcgacagttagaagacgttttattattagatcaaatgtcgaaaggtcgttttaattttggaaccgttcgagggctataccataaagattttcgagtatttggtgttgatatggaagagtctcgagcaattactcaaaatttctaccagatgataatggaaagcttacagacaggaaccattagctctgatagtgattacattcaatttcctaaggttgatgtatatcccaaagtgtactcaaaaaatgtaccaacccgtatgactgctgagtccgcaagtacgacagaatggctagcaatacaagggctaccaatggttcttagttggattattggtactaatgaaaaaaaagcacagatggaactctataatgaaattgcgacagaatatggtcatgatatatctaaaatagatcattgtatgacttatatttgttctgttgatgatgatgcacaaaaggcgcaagatgtttgtcgggagtttctgaaaaattggtatgactcatatgtaaatgcgaccaatatctttaatgatagcgatcaaactcgtggttatgattatcataaaggtcaatggcgtgattttgttttacaaggacatacaaacaccaatcgacgtgttgattatagcaatggtattaaccccgtaggcactcctgagcagtgtattgaaatcattcaacgtgatattgatgcaacgggtattacaaacattacatgcggatttgaagctaatggaactgaagatgaaataattgcttccatgcgacgctttatgacacaagtcgctcctttcttaaaagaacctaaataaattacttatttgatactagagataataaggaacaagttatgaaatttggattattttttctaaactttcagaaagatggaataacatctgaagaaacgttggataatatggtaaagactgtcacgttaattgattcaactaaatatcattttaatactgcctttgttaatgaacatcacttttcaaaaaatggtattgttggagcacctattaccgcagctggttttttattagggttaacaaataaattacatattggttcattaaatcaagtaattaccacccatcaccctgtacgtgtagcagaagaagccagtttattagatcaaatgtcagagggacgcttcattcttggttttagtgactgcgaaagtgatttcgaaatggaattttttagacgtcatatctcatcaaggcaacaacaatttgaagcatgctatgaaataattaatgacgcattaactacaggttattgccatccccaaaacgacttttatgattttccaaaggtttcaattaatccacactgttacagtgagaatggacctaagcaatatgtatccgctacatcaaaagaagtcgtcatgtgggcagcgaaaaaggcactgcctttaacgtttaagtgggaggataatttagaaaccaaagaacgctatgcaattctatataataaaacagcacaacaatatggtattgatatttcggatgttgatcatcaattaactgtaattgcgaacttaaatgctgatagaagtacggctcaagaagaagtgagagaatacttaaaagactatatcactgaaacttaccctcaaatggacagagatgaaaaaattaactgcattattgaagagaatgcagttgggtctcatgatgactattatgaatcgacaaaattagcagtggaaaaaacagggtctaaaaatattttattatcctttgaatcaatgtccgatattaaagatgtaaaagatattattgatatgttgaaccaaaaaatcgaaatgaatttaccataaggatcctaaaattaaaggcaatttctatattagattgcctttttggcgcgcctattctaatgcataataaatactgataacatcttatattttgtattatattttgtattatcgttgacatgtataattttgatatcaaaaactgattttccctctattattttcgagatttattttcttaattctctttaacaaactagaaatattgtatatacaaaaaattataaataatagatgaatagtttaattataggtgttcatcaatcgaaaaagcaacgtatcttatttaaagtgcgttgcttttttctcatttataaggttaaataattctcatatatcaagcaaagtgacagagctcggtactctcggcttgaacgaattggcggccgccctgcagtgatgatgagttggttcaaaagtatttagaagaggatggctgaatgacttcaaaactagttcatgtgaaagatgcagacaaaggctctgacatctactttgatccacagggccttgaaggcgccgtttttaattggaatggacagaaagattacagccaatacatttataacgctatgttgtatatgcgaagcggtagtttgatttgttgtgttgtgaatgacgatggcaagaagaagattcttgaacatgttcaggaagcaccataatgatgcaaaaaatccagcaggcaggggggatgtcaaaacttccaagcccttcgccgttggacaccgccccccatcgcacgcacaaaaaaaattccctctcagaaaaagttaaagcaaaaagttaaaatcaagttaaaggtagagcaatggcattaacagagaaaatggaaaaatttgctcttgccattgttgacggcaagacaaataaagaagcagcaatttcagcaggttatgcggaaaaaactgcatccgccgcaggtgctaggttagcaaaagatcctgaaattattgtctatattgaaatgttaaaggcccaaaaagaagggcgctctttaacatctaattcaccaaaagttaaacctaaagatacacccgaaaatagtggtgaagatgaaaaccctattgaggaatttcaatttgaaggtgatgatcctttagattttttaattaaggtcatgaacttcaatggcaacaagctgccacttagaatgcaagcagcaattgcagcattgccttataaacacgggaaggttgcggaaaaaggcaaaaaagaaactaaacaagacaaggcaaaggaagcgaccagaacaggaaaatacgccacattggacaatcagttgccaagctaactattaagaggaactttgcatgcaaagtctagaatacgaaaccgtaagtggtgaaactatcactattcaaaatatcaaagatggcccttgctgtcatgactctattgaaaagttgccagcaacagaaaggttggtgaagattacttatcaatgtcgcaagtgcttttccagattttccgaagaagattatcaattgattgttaatcaataaaaggttttgtatggatccgtaggcgatacggtgcgttggaggaaggaggaccacaactgccaacgtaataacccgctagcagtgggcgaaacagcgtagttaaagcaggggttcgcaacctgtcatacaaatttattccgccttcgggcggttttttcatggaccatttaaatgactgcaaaactaccagactggactacagcttgcccagactgggcgacccgtattgtttctaaacaatcgttaatgccgtgtaagccattattccccaaagtggctgacgtagcggagcgtatctttaaagagttaattcttgttgatgtgatgggtagccctaagatgggtgatgtcacattggaatgggtgatcgagtttgttcgtgcaatctttggcgcatatgatccaagcacaaagcgcagattaattcgtgaattctttcttttgatttcgaagaagaatactaaatctacgattgccgccggcattatgcttactgcattaattcttaatgatcgacaatctgccgaactaattattcttgcgcctactaaagaagttgctgataactcatttaatccaatccgggatttcatacgcgcagatgaagaattaagtgaaagatttaatgtatctgagcacacaaaaacagttacgcatctaggtaccggagcaacacttaaagttattgcagcagaatctaacgctgcagctggtaagaaagcttcaatcattttgatagatgaggtctggctattcgggaaacgtgccaacgctgaatcaatgttccgtgaagcaaagggtggtttagcatctcgtccagaaggttgtgtgatttatctgtctaccatgtcggatgaagtgccatgtggagtatttaagcagcttttagattatgccagagatgtacgtgacggaattaaagttgataaaagttttctaccacttatttatgaattccctaagcatcttgtagaagcaggcgaacatttaaaacctgaaaatttctacatcacaaacccaaacttgggtgcttcggttgatcttgaatatctgatttcggaatttaacaaagttaaagatgctagtgaagaatctcttagagacttcttggccaaacacttaaacattgaaatcggcatgaaccttcgtgctaaccggtgggcgggtgcagagtattggaatgctcaagctaaagatatccaaatcgaccaactaattgagctatccgatgtcattactttgggtattgatggcggtggtctcgacgacttacttggcttcgctgctttaggtcgtttaacagaagatcctcgtatctggtggctatggaatcatgcatgggcaaataagattgctttagagcgcagaaaagagaatgtgcctaagtatgaagacttcaagtctgagggttctctaactgttgttgaccgaataggcgatgacattgaccaactcgcagcaattgctaagaaggtttatgacagtggaaagcttaataagatcggactagatccattgggcttaggcggtcttttagatggcttacttgaggcaggaattccagaggaaagcatgtttgctgtgccacaaggctacaaactcatgtcctacatccttactactgagcgcaaattggcagaaggcaatctgtaccatgctggacaacagctaatgacttgggcggcaggtaatgcccgtgtcgtgatggtcggcaatggtatgcgaataaccaagcaagaatcaggtgttgggaagattgacccattgattgccacatttaacgcagttgctttgatgtcaagcaatcctgagcctgccaatcgcgttgatattgacgaatacttagaggatgtcgtgatagcatgagtaccacacaagagccggggttttggtcccgcttctggtcacgattgactggaaatacacaattaaaaaaaggcgattcgtcttatccatttgatagttatttgtcacccggtggatcggttgtcacaccagaaacagctttgaaactttccgcagtctgggcgtgtgtaaaattaagagctgaaactatctcaactcttcctttacagttgtacgacaacaataaacgtcttgctactgatcattacctttaccgtattttgcacgattcacccaatgccgatgatagctgacgaccttaaggataaatttctggtaaggaggacacgtatggaagtgggcaagttggggaagccgtatccgttgctgaatctggcatatgtgggagtataagacgcgcagcgtcgcatcaggcatttttttctgcgccaatgccggtagatagcaatccggcccgaggggcacaaaaaacccctcaagacccgtttagaggccccaaggggttatgctacgatactcaggcgccgggggcggtgtccggcggcccccagaggaactgcgccagttcctccggatcggtgaagccggagagatccagcggggtctcctcgaacacctcgaagtcgtgcaggaaggtgaaggcgagcagttcgcgggcgaagtcctcggtccgcttccactgcgccccgtcgagcagcgcggccaggatctcgcggtcgccccggaaggcgttgagatgcagttgcaccaggctgtagcgggggtctcccgcatagacgtcggtgaagtcgacgatcccggtgacctcggtcgcggccaggtccacgaagatgttggtcccgtgcaggtcgccgtggacgaaccggggttcgcggccggccagcagcgtgtccacgtccggcagccagtcctccaggcggtccagcagccggggcgagaggtagccccacccgcggtggtcctcgacggtcgccgcgcggcgttcccgcagcagttccgggaagacctcggaatggggggtgagcacggtgttcccggtcagcggcaccctgtgcagccggccgagcacccggccgagttcgcgggccagggcgagcagcgcgttccggtcggtcgtgccgtccatcgcggaccgccaggtggtgccggtcacccggctcatcaccaggtagggccacggccaggctccggtgccgggccgcagctcgccgcggacgaggaggcggggcaccggcaccggggcgtccgccaggaccgcgtacgcctccgactccgacgcgaggctctccggaccgcaccagtgctcgccgaacagcttgatcaccgggccgggctcgccgaccagtacggggttggtgctctcgccgggcacccgcagcaccggcggcaccggcagcccgagctcctccagggctcggcgggccagcggctcccagaattcctggtcgttccgcaggctcgcgtaggaatcatccgaatcaatacggtcgagaagtaacagggattcttgtgtcatatgaatcttactcctttgttaaattatttttgtttaagcattttgaatttgtatgcatcttagaacaatctcaaagttccaagctcttgcaagtctagatattgagtcaaaaaaaattttcaactcatactcttcctttttcaatattattgaagcatttatcagggttattgtctcatgagcgcacatttccccgaaaagtgccacctgacgtctaagaaaccagcggccgctcacactggctcaccttcgggtgggcctttctgcgtttatatactagagagagaatataaaaagccagattattaatccggcttttttattatttgggtgcctaatgagtgagctaactcacattaattgcgttgcgctcacgatcgtagaaatatctatgattatcttgaagaacgcaaccctatagcagctattgaaattgatgatttaattgaagaaaagacagatttacttgttgataatcgactgatggggcgcacaggcagacagaaagatactagggagttagtgatacatccgcattatgtggttgtatatgacatcactgatataatacggatactcagagtgctacacacatcgcaggagtggtcatgacttactcatgtactttggattatttagtgttataaaatcctgatttataaattttttttgttaaaaaagataaaagccccttgcaattgcttggggctttaccgtaatttatggggtacagatcttcgatactgacatatcggcaatcgaaagcattaaggtttgacgaccgctaatgatttcaccacaggggcttaatgtacctgtcttaaattctaaggttttaactcgctttgtcaagcatagaccccaaaaatttagccaatgtctgtaactcaatctgtccatgtgtgggtgatgaggtacagtgacgctagcacacatcggaaaaacgctattactaggggaactgaacagagtagcggacgcaatgagtagtcatttaattggcggttatgagcgtgttcaggcggtgctatcaatcgtaatcataacagtggcagcttgatacagtgatgtcatccctgatgcgaaagcgaccgaccgacggtacatcgaatgggaatactttagggtgatttttaagaatcgctctagggtgagtatttcccattcagctctgctccctccctctggtactttaatcaaaagcactactaaacatatgtttttaaataaaaaatattgatatagagataatattagtaagaataattaaacaattgaatatagataaatcattgttaaataaagattaattattaaaatgaatgtatacttatatataaatcaatgatttaaaatatttgataaagaaaacttttcaaaaaaaatataattgagattgtgtcatttcggtcaattcttaatatgttccacgcaagttttagctatggtgctaaacagaaatttgctgaaaaagaacttttcactgaactggttaaaatgtaagcagcctgagagccgccaaaaattttaaaaacaaaccgccttaatcatcttcaaaaaatacctctaaaacctcaccatttgcgttttaagacccatatttcatcctgcccttatgttcccatgctgatagctataaagtgtctgtaatcgcttcctatgacgttctaggctgttgataacttttggaacaacgcaaaatgttaaaatcctgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

(19) United States
(12) Patent Application Publication
(10) Pub. No.: US 2022/0090094 A1
(43) Pub. Date: Mar. 24, 2022

(54) NON-REPLICATIVE TRANSDUCTION PARTICLES AND TRANSDUCTION PARTICLE-BASED REPORTER SYSTEMS FOR DETECTION OF ACINETOBACTER BAUMANNII

(71) Applicant: Roche Molecular Systems, Inc., Pleasanton, GA (US)

(72) Inventors: Kathleen Y. Dunphy, Danville, CA (US); Xiaowen Liu, San Ramon, CA (US); Letong Jia, Cupertino, CA (US); Jeffrey Alexander, Hayward, CA (US); Xun Zhuang, Pleasanton, CA (US)

(21) Appl. No.: 17/418,027

(22) PCT Filed: Dec. 22, 2019
(86) PCT No.: PCT/EP2019/086887

§ 371 Date: Jun. 24, 2021

Related U.S. Application Data
(60) Provisional application No. 62/899,985, filed on Sep. 13, 2019.
(60) Provisional application No. 62/785,510, filed on Dec. 27, 2018.

(51) Int. Cl. C12N 15/74; C12Q 1/04; C12N 7/00

(52) U.S. Cl. CPC C12N 15/74; C12Q 1/04; C12N 2795/10321; C12N 2795/10121; C12N 7/00

(57) ABSTRACT

The present invention relates to novel bacteriophages that are specific for Acinetobacter baumannii (A. baumannii) and to methods for producing non-replicative transduction particles (NRTPs) derived from these bacteriophages and to the use of the NRTPs for detection of A. baumannii.

### BACKGROUND OF THE INVENTION

### Field of the Invention

The invention relates to methods and compositions for packaging and delivery of non-replicative transduction reporter molecules for detecting target cells.

### CROSS REFERENCE TO RELATED APPLICATIONS

This application is a U.S. national stage filing under 35 U.S.C. § 371 of International Application No. PCT/EP2019/086887, filed Dec. 22, 2019, entitled “NON-REPLICATIVE TRANSDUCTION PARTICLES AND TRANSDUCTION PARTICLE-BASED REPORTER SYSTEMS FOR DETECTION OF ACINETOBACTER BAUMANNII”, which claims the benefit of priority to U.S. Provisional Patent Application No. 62/785,510, filed on Dec. 27, 2018, and to U.S. Provisional Patent Application No. 62/899,985 filed on Sep. 13, 2019, each of which is hereby incorporated in its entirety by reference.

### REFERENCE TO SEQUENCE LISTING

This application contains a Sequence Listing submitted as an electronic text file named “P35222-WO PCT filing sequence listing”, having a size in bytes of 46 kb, and created on Nov. 20, 2019.

### Description of the Related Art

A transduction particle refers to a virus capable of delivering a non-viral nucleic acid into a cell. Viral-based reporter systems have been used to detect the presence of cells and rely on the lysogenic phase of the virus to allow expression of a reporter molecule from the cell. These viral-based reporter systems use replication-competent transduction particles that express reporter molecules and cause a target cell to emit a detectable signal.

However, the lytic cycle of the virus has been shown to be deleterious to viral-based reporter assays. Carrière, C. et al., Conditionally replicating luciferase reporter phages: Improved sensitivity for rapid detection and assessment of drug susceptibility of Mycobacterium tuberculosis. Journal of Clinical Microbiology, 1997. 35(12): p. 3232-3239. Carrière et al. developed M. tuberculosis/bacillus Calmette-Guérin (BCG) luciferase reporter phages that have their lytic cycles suppressed at 30° C., but active at 37° C. Using this system, Carrière et al. have demonstrated the detection of BCG using phage reporters with a suppressed lytic cycle.

There are disadvantages, however, associated with suppressing but not eliminating the replication functions of the bacteriophage in bacteriophage-based reporter assays. First, controlling replication functions of the bacteriophage imposes limiting assay conditions. For example, the lytic cycle of the reporter phage phAE40 used by Carrière et al. was repressed when the phage was used to infect cells at the non-permissive temperature of 30° C. This temperature requirement imposed limiting conditions on the reporter assay in that the optimum temperature for the target bacteria was 37° C. These limiting conditions hinder optimum assay performance.

Moreover, the replication functions of the virus are difficult to control. The replication of the virus should be suppressed during the use of the transduction particles as a reporter system. For example, the lytic activity of the reporter phage phAE40 reported by Carrière et al. was reduced but was not eliminated, resulting in a drop in luciferase signal in the assay. Carrière et al. highlighted possible causes for the resulting drop in reporter signal, such as intact phage-expressed genes and temperature limitations of the assay, all stemming from the fact that the lytic cycle of the phage reporter was not eliminated.

Reporter assays relying on the natural lysogenic cycle of phages can be expected to exhibit lytic activity sporadically. In addition, assays that rely on the lysogenic cycle of the phage can be prone to superinfection immunity from target cells already lysogenized with a similar phage, as well as naturally occurring host restriction systems that target incoming virus nucleic acid, thus limiting the host range of these reporter phages.

In other examples, transduction particle production systems are designed to package exogenous nucleic acid molecules, but the transduction particle often contains a combination of exogenous nucleic acid molecules and native progeny virus nucleic acid molecules. The native virus can exhibit lytic activity that is a hindrance to assay performance, and the lytic activity of the virus must be eliminated to purify transduction particles. However, this purification is generally not possible. In U.S. 2009/0155768 A, entitled Reporter Plasmid Packaging System for Detection of Bacteria, Scholl et al. describes the development of such a transduction particle system. The product of the system is a combination of reporter transduction particles and native bacteriophage (FIG. 8 in the reference). Although the authors indicate that the transduction particle and native bacteriophage can be separated by ultracentrifugation, this separation is only possible in a system where the transduction particle and the native virus exhibit different densities that would allow separation by ultracentrifugation. While this characteristic is exhibited by the bacteriophage T7-based packaging system described in the reference, this is not a characteristic that is generally applicable for other virus systems. It is common for viral packaging machinery to exhibit headful packaging that would result in native virus and transduction particles to exhibit indistinguishable densities that cannot be separated by ultracentrifugation. Virus packaging systems also rely on a minimum amount of packaging as a requirement for proper virus structural assembly that results in native virus and transduction particles with indistinguishable densities.

Thus, there is a need for non-replicative transduction particles that do not suffer from the deleterious effects from lytic functions of the virus and the possibility of being limited by superinfection immunity and host restriction mechanisms that target virus nucleic acid molecules and viral functions, all of which can limit the performance of the reporter assay by increasing limits of detection and resulting in false negative results.

Even where transduction particles have been engineered, methods for using the transduction particles to detect and report the presence of target nucleic acid molecules in cells have limitations. Some methods require disruption of the cell and cumbersome techniques to isolate and detect transcripts in the lysate. Detection methods include using labeled probes such as antibodies, aptamers, or nucleic acid probes. Labeled probes directed to a target gene can result in non-specific binding to unintended targets or generate signals that have a high signal-to-noise ratio. Therefore, there is a need for specific, effective and accurate methods for detection and reporting of endogenous nucleic acid molecules in cells.

More recently, methods and systems for packaging reporter nucleic acid molecules into non-replicative transduction particles (NRTPs), also referred herein as Smarticles, have been described in U.S. Pat. No. 9,388,453 (incorporated herein by reference in its entirety) in which the production of replication-competent native progeny virus nucleic acid molecules were greatly reduced due to the disruption of the packaging initiation site in the bacteriophage genome.

Acinetobacter baumannii (A. baumannii) is a Gram-negative coccobacillus that has become increasingly problematic as a major cause of nosocomial infections and global epidemics. Infection by A. baumannii may result in septicemia, ventilator-associated pneumonia, urinary tract infections, and wound infections (Beggs et al., 2006; Peleg et al. 2008) with immunocompromised individuals at particular risk. The A. baumannii strains causing infections are often extensively resistant to antibiotics and pose a serious public health threat, which prompted the World Health Organization recently to declare it the critical-level ‘priority 1’ pathogen on the list of developing new antibiotics targeting it (WHO, 2017). Furthermore, mortality rates are particularly high with A. baumannii infections; in patients with ventilator-associated pneumonia and bloodstream infections, mortality rates were as high as 35% (Antunes et al., 2014). One risk factor for the high mortality rates observed with A. baumannii infection stem from inappropriate antibiotic treatment (Lemos E V et al., 2014).

Rapid diagnosis of A. baumannii is critical for identifying appropriate antibiotic therapy and controlling the spread of infection in a clinical setting. Current commercially available methods for detecting A. baumannii infections include phenotypic methods (e.g., VITEK 2, Biomerieux) and DNA-based methods (e.g., PCR amplification of 16s rRNA) (Li P, et al., 2015). However, a need exists for assays that can rapidly detect the presence of A. baumannii in biological samples without requiring the use of native phages, which must infect the host bacteria to complete the lytic life cycle and also encounter bacterial host defense mechanisms.

### SUMMARY OF THE INVENTION

The present invention relates to compositions comprising novel bacteriophages specific to A. baumannii that have broad host range within this species. In one embodiment, the novel bacteriophage is Abi 33, which belongs in the Myoviridae family. In another embodiment, the novel bacteriophage is Abi 49 or Abi 147, which belong in the Siphoviridae family. The present invention also relates to the production of non-replicative transduction particles (NRTPs) that exhibit specificity for A. baumannii that are derived from the genomes of these novel bacteriophages. Thus, the present invention relates to a composition comprising a bacteriophage genome, wherein the bacteriophage genome is derived from a bacteriophage selected from the group consisting of Abi 33, Abi 49 and Abi 147 and wherein the bacteriophage genome contains a disruption of one or more genes that encode packaging-related enzymatic activity. In one embodiment, the one or more genes that encode packaging-related enzymatic activity comprises a terS gene, a terL gene or both terS and terL genes. In one embodiment, the disruption of the one or more genes that encode packaging-related enzymatic activity comprises a deletion of a nucleotide sequence selected from SEQ ID NO: 1, SEQ ID NO: 2 or SEQ ID NO: 3.

The present invention also relates to a bacterial cell packaging system for packaging a reporter plasmid comprising a reporter gene into a Smarticles non-replicative transduction particle (NRTP) for introduction into an A. baumannii cell. In one embodiment, the packaging system comprises a host A. baumannii cell, a first nucleic acid construct inside the host A. baumannii cell comprising or consisting of a bacteriophage genome having a disruption of one or more genes that encode packaging-related enzymatic activity, wherein the disruption prevents packaging of the bacteriophage genome into the NRTP, and wherein the bacteriophage genome is selected from the group consisting of the genome of bacteriophage Abi 33, the genome of bacteriophage Abi 49, and the genome of bacteriophage 147, and a second nucleic acid construct inside the host A. baumannii cell, which is separate from the first nucleic acid construct, said second nucleic acid construct comprising a reporter nucleic acid molecule having a reporter gene and one or more genes that encode packaging-related enzymatic activity that complements the disruption on the bacteriophage genome and facilitates packaging of a replicon of the reporter nucleic acid molecule into the NRTP. In one embodiment, the one or more genes that encode packaging-related enzymatic activity comprises a terS gene, a terL gene or both terS and terL genes. In one embodiment, the disruption of the one or more genes that encode packaging-related enzymatic activity comprises a deletion of a nucleotide sequence selected from SEQ ID NO: 1, SEQ ID NO: 2 or SEQ ID NO: 3. In some embodiments, the disruption is via deletion, insertion, mutation, or replacement. In another embodiment, the reporter nucleic acid molecule comprises a nucleotide sequence selected from SEQ ID NO: 4, SEQ ID NO: 5 or SEQ ID NO: 6.

The present invention also relates to a method of producing NRTPs from the aforementioned bacterial cell packaging system comprising inducing a lytic phase of the bacterial cell packaging system and allowing the replicon of the reporter molecule to be packaged to produce the NRTPs. In one embodiment, the one or more genes that encode packaging-related enzymatic activity comprises a terS gene, a terL gene or both terS and terL genes. In one embodiment, the disruption of the one or more genes that encode packaging-related enzymatic activity comprises a deletion of a nucleotide sequence selected from SEQ ID NO: 1, SEQ ID NO: 2 or SEQ ID NO: 3. In another embodiment, the reporter nucleic acid molecule comprises a nucleotide sequence selected from SEQ ID NO: 4, SEQ ID NO: 5 or SEQ ID NO: 6.

The present invention also relates to methods of detecting A. baumannii in a sample comprising the steps of providing NRTPs derived from bacteriophage Abi 33, NRTPs derived from bacteriophage Abi 49, NRTPs derived from bacteriophage Abi 147, or any combination of the above that are produced by the aforementioned NRTP production method to the sample, providing conditions for the reporter gene to produce a detectable signal, and detecting the presence or absence of the detectable signal to indicate the presence or absence of A. baumannii. In one embodiment, the method comprises a step before or after providing NRTPs to the sample of providing an antimicrobial agent to the sample and detecting for the presence or absence of the detectable signal to indicate whether the sample contains A. baumannii that is resistant or susceptible to the antimicrobial agent. In one embodiment, the NRTPs comprise a reporter nucleic acid molecule that comprises a nucleotide sequence selected from SEQ ID NO: 4, SEQ ID NO: 5 or SEQ ID NO: 6.

### BRIEF DESCRIPTION OF THE DRAWINGS

FIG. 1a shows TEM images of Abi 33 lysate containing 2 populations of Myoviridae phages.

FIG. 1b shows a TEM image of Abi 49 lysate containing Siphoviridae phages.

FIG. 1c shows a TEM image of Abi 147 lysate containing Siphoviridae phages.

FIG. 2 shows a schematic of Smarticles non-replicative transduction particle (NRTP) technology using a deletion/complementation strategy. Each of the candidate phages were engineered to delete the pac site/pac function, and then complemented back by plasmid carrying package-related DNA sequences and genes. The complementary plasmids also carry luciferase as a reporter gene. Smarticle NRTPs are generated from engineered prophage harboring packaging/reporter plasmids.

FIG. 3 is a representative schematic of a packaging/reporter plasmid for the production of Abi 33, Abi 49, and Abi 147 NRTPs.

FIG. 4a shows a Gram (−) strain layout for A. baumannii cross-reactivity Relative-Light Units (RLU) assays. Strain shorthand is indicated as follows Kpn: K. pneumoniae, Eco: E. coli, Kox: K. oxytoca, Eae: E. aerogenes, Ed: E. cloacae, Cfi: C. freundii, Cko: C. koseri, Sms: S. marcescens, Pae: P. aeruginosa, Pms: P. mirabilis, Abi: A. baumannii.

FIG. 4b shows the RLU-based coverage of Abi 33, Abi 49, and Abi 147 Smarticles NRTPs tested for cross-reactivity against Enterobacteriaceae and other Gram (−) bacteria in FIG. 4a. No RLU-positive results were observed. Peaks in C1 and G1 are injection spikes and not true positive signals. The RLU-positive A. baumannii strains in column 12 served as positive controls in the assay to indicate typical Abi 49 titers.

FIG. 5 is a representative schematic of Abi packaging/reporter plasmid pZX057.

FIG. 6 is a representative schematic of the 9247 bp plasmid pZX058 that was derived from Abi 33 to generate new Abi 33 Smarticles NRTPs.

FIGS. 7-I, 7-II, 7-III and 7-IV show the annotated nucleotide sequence of plasmid pZX058 (SEQ ID NO: 4).

FIG. 8 is a representative schematic of the 9464 bp plasmid pZX065 that was derived from Abi 49 to generate new Abi 49 Smarticles NRTPs.

FIGS. 9-I, 9-II, 9-III and 9-IV show the annotated nucleotide sequence of plasmid pZX065 (SEQ ID NO. 5).

FIG. 10 is a representative schematic of the 9768 bp plasmid pZX066 that was derived from Abi 147 to generate new Abi 147 Smarticles NRTPs.

FIGS. 11-I, 11-II, 11-III and 11-IV show the annotated nucleotide sequence of plasmid pZX066 (SEQ ID NO: 6).

### DETAILED DESCRIPTION OF THE INVENTION

### I. Definitions

Terms used in the claims and specification are defined as set forth below unless otherwise specified. As used herein, “reporter nucleic acid molecule” refers to a nucleotide sequence comprising a DNA or RNA molecule. The reporter nucleic acid molecule can be naturally occurring or an artificial or synthetic molecule. In some embodiments, the reporter nucleic acid molecule is exogenous to a host cell and can be introduced into a host cell as part of an exogenous nucleic acid molecule, such as a plasmid or vector. In certain embodiments, the reporter nucleic acid molecule can be complementary to a target gene in a cell. In other embodiments, the reporter nucleic acid molecule comprises a reporter gene encoding a reporter molecule (e.g., reporter enzyme, protein). In some embodiments, the reporter nucleic acid molecule is referred to as a “reporter construct” or “nucleic acid reporter construct.”

A “reporter molecule” or “reporter” refers to a molecule (e.g., nucleic acid or protein) that confers onto an organism a detectable or selectable phenotype. The detectable phenotype can be colorimetric, fluorescent or luminescent, for example. Reporter molecules can be expressed from reporter genes encoding enzymes mediating luminescence reactions (LuxA, LuxB, LuxAB, Luc, Ruc, nLuc), genes encoding enzymes mediating colorimetric reactions (LacZ, HRP), genes encoding fluorescent proteins (GFP, eGFP, YFP, RFP, CFP, BFP, mCherry, near-infrared fluorescent proteins), nucleic acid molecules encoding affinity peptides (His-tag, 3×-FLAG), and genes encoding selectable markers (e.g. ampC, tet(M), zeoR, hph, CAT, erm). The reporter molecule can be used as a marker for successful uptake of a nucleic acid molecule or exogenous sequence (plasmid) into a cell. The reporter molecule can also be used to indicate the presence of a target gene, target nucleic acid molecule, target intracellular molecule, or a cell, as described herein. Alternatively, the reporter molecule can be a nucleic acid, such as an aptamer or ribozyme. In some aspects of the invention, the reporter nucleic acid molecule is operatively linked to a promoter. In other aspects of the invention, the promoter can be chosen or designed to contribute to the reactivity and cross-reactivity of the reporter system based on the activity of the promoter in specific cells (e.g., specific species) and not in others. In certain aspects, the reporter nucleic acid molecule comprises an origin of replication. In other aspects, the choice of origin of replication can similarly contribute to reactivity and cross-reactivity of the reporter system, when replication of the reporter nucleic acid molecule within the target cell contributes to or is required for reporter signal production based on the activity of the origin of replication in specific cells (e.g., specific species) and not in others. In some embodiments, the reporter nucleic acid molecule forms a replicon capable of being packaged as concatameric DNA into a progeny virus during virus replication.

As used herein, a “target transcript” refers to a portion of a nucleotide sequence of a DNA sequence or an mRNA molecule that is naturally formed by a target cell including that formed during the transcription of a target gene and mRNA that is a product of RNA processing of a primary transcription product. The target transcript can also be referred to as a cellular transcript or naturally occurring transcript.

As used herein, the term “transcript” refers to a length of nucleotide sequence (DNA or RNA) transcribed from a DNA or RNA template sequence or gene. The transcript can be a cDNA sequence transcribed from an RNA template or an mRNA sequence transcribed from a DNA template. The transcript can be protein coding or non-coding. The transcript can also be transcribed from an engineered nucleic acid construct.

A transcript derived from a reporter nucleic acid molecule can be referred to as a “reporter transcript.” The reporter transcript can include a reporter sequence and a cis-repressing sequence. The reporter transcript can have sequences that form regions of complementarity, such that the transcript includes two regions that form a duplex (e.g., an intermolecular duplex region). One region can be referred to as a “cis-repressing sequence” and has complementarity to a portion or all of a target transcript and/or a reporter sequence. A second region of the transcript is called a “reporter sequence” and can have complementarity to the cis-repressing sequence. Complementarity can be full complementarity or substantial complementarity. The presence and/or binding of the cis-repressing sequence with the reporter sequence can form a conformation in the reporter transcript, which can block further expression of the reporter molecule. The reporter transcript can form secondary structures, such as a hairpin structure, such that regions within the reporter transcript that are complementary to each other can hybridize to each other.

“Introducing into a cell,” when referring to a nucleic acid molecule or exogenous sequence (e.g., plasmid, vector, construct), means facilitating uptake or absorption into the cell, as is understood by those skilled in the art. Absorption or uptake of nucleic acid constructs or transcripts can occur through unaided diffusive or active cellular processes, or by auxiliary agents or devices including via the use of bacteriophage, virus, and transduction particles. The meaning of this term is not limited to cells in vitro; a nucleic acid molecule may also be “introduced into a cell,” wherein the cell is part of a living organism. In such instance, introduction into the cell will include the delivery to the organism. For example, for in vivo delivery, nucleic acid molecules, constructs or vectors of the invention can be injected into a tissue site or administered systemically. In vitro introduction into a cell includes methods known in the art, such as electroporation and lipofection. Further approaches are described herein or known in the art.

A “transduction particle” refers to a virus capable of delivering a non-viral nucleic acid molecule into a cell. The virus can be a bacteriophage, adenovirus, etc.

A “non-replicative transduction particle” or “NRTP” refers to a virus capable of delivering a non-viral nucleic acid molecule into a cell, but is incapable of packaging its own replicated viral genome into the transduction particle. The virus can be a bacteriophage, adenovirus, etc.

A “plasmid” is a small DNA molecule that is physically separate from, and can replicate independently of, chromosomal DNA within a cell. Most commonly found as small circular, double-stranded DNA molecules in bacteria, plasmids are sometimes present in archaea and eukaryotic organisms. Plasmids are considered replicons, capable of replicating autonomously within a suitable host.

A “vector” is a nucleic acid molecule used as a vehicle to artificially carry foreign genetic material into another cell, where it can be replicated and/or expressed.

A “virus” is a small infectious agent that replicates only inside the living cells of other organisms. Virus particles (known as virions) include two or three parts: i) the genetic material made from either DNA or RNA molecules that carry genetic information; ii) a protein coat that protects these genes; and in some cases, iii) an envelope of lipids that 9388

As used herein, the term “complement” refers to a non-disrupted sequence that is in the presence of an identical sequence that has been disrupted, or to the relationship of the non-disrupted sequence to the disrupted sequence. In one embodiment, the complement comprises a gene encoded on a polynucleotide in a cell that is functional and capable of expression, and expresses a protein with the same function as a disrupted gene on a bacteriophage prior to disruption. In some embodiments, the complement gene has greater than 80%, 85%, 90%, 91%, 92%, 93%, 94%, 95%, 96%, 97%, 98%, or 99% sequence identity to the disrupted bacteriophage gene prior to disruption, i.e., the native bacteriophage gene. In some embodiments, the complement gene is identical to the disrupted bacteriophage gene prior to disruption, i.e., the native bacteriophage gene. In some embodiments, the complement gene comprises a polynucleotide sequence that has been deleted from the bacteriophage. In some embodiments, the complement gene refers to a gene encoding packaging machinery of a bacteriophage on a plasmid, where the same gene has been disrupted in a bacteriophage. Thus, the plasmid is required to be in the presence of a bacteriophage with a mutated packaging machinery gene to provide the necessary packaging machinery necessary for packaging a polynucleotide into a transduction particle.

As used herein, the term “packaging-related enzymatic activity” refers to one or more polypeptides crucial for the interaction with a packaging initiation site sequence to package a polynucleotide into a transduction particle. In some embodiments, a pair of terminase genes is required for such an interaction, wherein each terminase encodes a packaging-related enzymatic activity. In some embodiments, the enzymatic activity is encoded by a terS and/or terL gene from A. baumannii bacteriophages that were discovered in the present invention. In these embodiments, each of the pair of terminase genes express a packaging-related enzymatic activity, and a functional version of both are required for packaging of a polynucleotide with the packaging initiation site. In some embodiments, disruption of one of the genes of a plurality of genes associated with a packaging-related enzymatic activity eliminates the packaging-related enzymatic activity. In some embodiments, both of the pair of terminase genes are disrupted on the bacteriophage genome, thus disrupting the entire set of packaging-related enzymatic activity encoding genes on the bacteriophage.

The term “ameliorating” refers to any therapeutically beneficial result in the treatment of a disease state, e.g., a disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines. “G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

As used herein, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Complementary sequences are also described as binding to each other and characterized by binding affinities.

For example, a first nucleotide sequence can be described as complementary to a second nucleotide sequence when the two sequences hybridize (e.g., anneal) under stringent hybridization conditions. Hybridization conditions include temperature, ionic strength, pH, and organic solvent concentration for the annealing and/or washing steps. The term stringent hybridization conditions refers to conditions under which a first nucleotide sequence will hybridize preferentially to its target sequence, e.g., a second nucleotide sequence, and to a lesser extent to, or not at all to, other sequences. Stringent hybridization conditions are sequence dependent, and are different under different environmental parameters. Generally, stringent hybridization conditions are selected to be about 5° C. lower than the thermal melting point (Tₘ) for the nucleotide sequence at a defined ionic strength and pH. The Tₘ is the temperature (under defined ionic strength and pH) at which 50% of the first nucleotide sequences hybridize to a perfectly matched target sequence. An extensive guide to the hybridization of nucleic acids is found in, e.g., Tijssen (1993) Laboratory Techniques in Biochemistry and Molecular Biology—Hybridization with Nucleic Acid Probes part I, chap. 2, “Overview of principles of hybridization and the strategy of nucleic acid probe assays,” Elsevier, N.Y. (“Tijssen”). Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, provided the above requirements with respect to their ability to hybridize are fulfilled. Such non-Watson-Crick base pairs includes, but not limited to, G:U Wobble or Hoogstein base pairing.

The terms “complementary,” “fully complementary” and “substantially complementary” herein may be used with respect to the base matching between two strands of a dsRNA, or between the antisense strand of a dsRNA and a target sequence, between complementary strands of a single stranded RNA sequence or a single stranded DNA sequence, as will be understood from the context of their use.

As used herein, a “duplex structure” comprises two anti-parallel and substantially complementary nucleic acid sequences. Complementary sequences in a nucleic acid construct, between two transcripts, between two regions within a transcript, or between a transcript and a target sequence can form a “duplex structure.” In general, the majority of nucleotides of each strand are ribonucleotides, but as described in detail herein, each or both strands can also include at least one non-ribonucleotide, e.g., a deoxyribonucleotide and/or a modified nucleotide. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the duplex minus any overhangs that are present in the duplex. Generally, the duplex structure is between 15 and 30 or between 25 and 30, or between 18 and 25, or between 19 and 24, or between 19 and 21, or 19, 20, or 21 base pairs in length. In one embodiment the duplex is 19 base pairs in length. In another embodiment the duplex is 21 base pairs in length. When two different siRNAs are used in combination, the duplex lengths can be identical or can differ.

As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to produce a detectable signal from a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### II. Lysogenic and Lytic Cycle of Viruses

Viruses undergo lysogenic and lytic cycles in a host cell. If the lysogenic cycle is adopted, the phage chromosome can be integrated into the bacterial chromosome, or it can establish itself as a stable plasmid in the host, where it can remain dormant for long periods of time. If the lysogen is induced, the phage genome is excised from the bacterial chromosome and initiates the lytic cycle, which culminates in lysis of the cell and the release of phage particles. The lytic cycle leads to the production of new phage particles, which are released by lysis of the host.

In addition, virus-based reporter assays, such as phage-based reporters, can suffer from limited reactivity (i.e., analytical inclusivity) due to limits in the phage host range caused by host-based and prophage-derived phage resistance mechanisms. These resistance mechanisms target native phage nucleic acid that can result in the degradation or otherwise inhibition of the phage DNA and functions. Such resistance mechanisms include restriction systems that cleave phage DNA and CRISPR systems that inhibit phage-derived transcripts.

Both lytic activity and phage resistance can be inhibitory to assays based on reporter phages. Lytic activity can inhibit signal by destroying or otherwise inhibiting the cell in its ability to generate a detectable signal and thus affecting limits of detection by reducing the amount of detectable signal or preventing the generation of a detectable signal. Phage resistance mechanisms can limit the host range of the phage and limit the inclusivity of the phage-based reporter, similarly affecting limits of detection by reducing the amount of detectable signal or preventing the generation of a detectable signal. Both lytic activity and phage resistance caused by the incorporation of phage DNA in a reporter phage can lead to false-negative results in assays that incorporate these phage reporters.

### III. Methods for Producing Non-Replicative Transduction Particles (NRTP)

Disruption/Complementation-Based Methods for Producing Non-Replicative Transduction Particles.

Disclosed herein are non-replicative transduction particle packaging systems, referred herein also as Smarticles systems, based on disruption of a component of the genome of a virus that is recognized by the viral packaging machinery as the element from which genomic packaging is initiated during viral production. In an embodiment, this disruption disrupts a packaging initiation site from a bacteriophage, and also disrupts a terminase function. Examples of the disrupted elements include the pac-site sequence of pac-type bacteriophages and the cos-site sequence of cos-type bacteriophages. When the packaging initiation site sequence within the phage is disrupted, the phage cannot produce functional terminases. In an example, the pac-site is encoded within a pacA gene sequence, and terminase functions require both a functional PacA and PacB. Plasmid DNA is packaged into a phage capsid by complementing said disrupted terminases and including a recognizable packaging initiation site on the plasmid DNA.

Packaging initiation sites are often found within coding regions of genes that are essential to virus production. A region of the bacteriophage genome can be disrupted by an insertion, replacement, deletion, or mutation that disrupts the packaging initiation site. Examples of disruptions that accomplish this include, but are not limited to, an allelic exchange event that replaces a sequence on the bacteriophage genome that contains the packaging initiation site sequence with another sequence such as that of an antibiotic resistance gene, or the complete deletion of the small and large terminase genes. In an example employing the terminase genes pacA and pacB, pacA can be disrupted in a manner that causes polar effects that also disrupt pacB expression and/or overall terminase function mediated by PacA and PacB. Other examples can include the disruption of terminase genes and can also include terS and terL genes from A. baumannii bacteriophages discovered in the present invention

In one example, a cell's genome is lysogenized with a viral genome where the packaging initiation site has been disrupted. The cell can be Gram-negative or Gram-positive. A complementing plasmid (or reporter nucleic acid molecule) is introduced into the cell, and the plasmid DNA includes at least the gene that has been disrupted in the bacteriophage, as well as the packaging initiation site sequence, and optionally additional bacteriophage genes and a reporter gene, which can encode a detectable and/or a selectable marker. The plasmid can be constructed using methods found in U.S. Pat. No. 9,388,453, hereby incorporated by reference in its entirety. One or more genes of the plasmid can be operatively linked to a promoter, such as an inducible promoter (which can be induced when packaging is initiated by inducing the bacteriophage). In some embodiments, the promoter can be a native promoter of a small terminase gene (terS) or a large terminase (terL) gene. The native promoter can be controlled by the bacteriophage, and thus effectively acts as a conditional promoter induced during packaging.

In some examples, it is preferable that the disruption/complementation is designed such that there is no homology between the mutated virus DNA and the complementing exogenous DNA. This is because lack of homology between the mutated virus DNA and the complementing exogenous DNA avoids the possibility of homologous recombination between the two DNA molecules that can result in re-introduction of a packaging sequence into the virus genome. To accomplish a lack of homology, one strategy is to delete the entire gene (or genes) that contains the packaging initiation site sequence from the virus genome and then complement this gene with an exogenous DNA molecule that preferably contains no more than exactly the DNA sequence that was deleted from virus. In this strategy, the complementing DNA molecule is designed to express the gene that was deleted from the virus. Another example of such a system is provided using the bacteriophage φ80α, a pac-type phage. The phage genome is lysogenized in a host bacterial cell, and the phage genome includes a small terminase gene where the pac-site of a pac-type prophage φ80α has been deleted. A plasmid including a complementary small terminase gene with a native pac-site is transformed into the cell. When the lytic cycle of the lysogenized prophage is induced, the bacteriophage packaging system packages plasmid DNA into progeny bacteriophage structural components, rather than packaging the native bacteriophage DNA. The packaging system thus produces non-replicative transduction particles carrying plasmid DNA.

Phages are the most abundant life form in the biosphere (Clokie et al., 2011), and 70% of sequenced bacterial genomes contain prophage-like structures (Chen, et al., 2006). Touchon, et al., observed that complete prophages (i.e., phages integrated into bacterial genomes) were predominant when analyzing the bacterial genomes of 133 Acinetobacter spp. (Touchon et al., 2014) This natural abundance of prophages in nature was utilized to isolate de novo phages specific to A. baumannii with broad host range within this species. These phages were converted into non-replicative transduction particles that yielded good inclusivity and exclusivity in luminescence assays.

Given the extensive genetic diversity and genomic plasticity of A. baumannii strains (Sahl, J W et al., 2015; Snitkin et al., 2011), non-replicative transduction particles were combined as a cocktail in a single assay to account for potential diversity in phage receptors on the A. baumannii cells. In doing so, inclusivity of A. baumannii strains in the assay improved without interfering effects. This approach was taken in the same way that phage cocktails are used therapeutically in phage therapy (Chan, B K et al., 2013). The advantage of this technology is that the assay requires only DNA delivery and luminescence reaction to occur in a short period of time without the phage having to complete the lytic life cycle or encounter bacterial host defense mechanisms.

The reporter gene encodes a detectable marker or a selectable marker. In an example, the reporter gene is selected from the group consisting of enzymes mediating luminescence reactions (LuxA, LuxB, LuxAB, Luc, Ruc, nLuc), enzymes mediating colorimetric reactions (LacZ, HRP), fluorescent proteins (GFP, eGFP, YFP, RFP, CFP, BFP, mCherry, near-infrared fluorescent proteins), affinity peptides (His-tag, 3×-FLAG), and selectable markers (ampC, tet(M), CAT, erm). In an embodiment, the reporter gene is luxA. In some embodiments, the resistance marker comprises an antibiotic resistance gene. In some embodiments, the resistance marker is a kanamycin resistance gene (kan). In some embodiments, the constitutive promoter comprises pBla (promoter for ampicillin resistance gene). In some embodiments, the bacteriophage genome disruption is accomplished by an allelic exchange event that replaces or disrupts a sequence on the bacteriophage genome that contains the packaging initiation site sequence.

In an example, a pair of terminase genes on a bacteriophage genome, e.g., terS and terL, can be disrupted in a manner that causes polar effects that also disrupt expression of one of the terminase genes and/or overall terminase function mediated by the terminase genes. The disrupted bacteriophage can be complemented with a plasmid comprising terminase genes, e.g., terS and terL, of the bacteriophage genome. When the mutated virus is undergoing a lytic cycle, the viral packaging proteins, produced either from the bacteriophage genome or (if disrupted) the complementing plasmid, package a replicon of the plasmid DNA into the packaging unit because it contains a packaging initiation site, and non-replicative transduction particles are produced carrying the replicated plasmid DNA.

### VI. Reporters

In some embodiments, the NRTPs and constructs of the invention comprise a reporter nucleic acid molecule including a reporter gene. The reporter gene can encode a reporter molecule, and the reporter molecule can be a detectable or selectable marker. In certain embodiments, the reporter gene encodes a reporter molecule that produces a detectable signal when expressed in a cell.

In certain embodiments, the reporter molecule can be a fluorescent reporter molecule, such as, but not limited to, a green fluorescent protein (GFP), enhanced GFP, yellow fluorescent protein (YFP), cyan fluorescent protein (CFP), blue fluorescent protein (BFP), red fluorescent protein (RFP) or mCherry, as well as near-infrared fluorescent proteins.

In other embodiments, the reporter molecule can be an enzyme mediating luminescence reactions (LuxA, LuxB, LuxAB, Luc, Ruc, nLuc, etc.). Reporter molecules can include a bacterial luciferase, a eukaryotic luciferase, an enzyme suitable for colorimetric detection (LacZ, HRP), a protein suitable for immunodetection, such as affinity peptides (His-tag, 3×-FLAG), a nucleic acid that function as an aptamer or that exhibits enzymatic activity (ribozyme), or a selectable marker, such as an antibiotic resistance gene (ampC, tet(M), CAT, erm). Other reporter molecules known in the art can be used for producing signals to detect target nucleic acids or cells.

In other aspects, the reporter molecule comprises a nucleic acid molecule. In some aspects, the reporter molecule is an aptamer with specific binding activity or that exhibits enzymatic activity (e.g., aptazyme, DNAzyme, ribozyme).

Reporters and reporter assays are described further in Section V herein.

### V. NRTPs and Reporter Assays

Inducer Reporter Assay

In some embodiments, the invention comprises methods for the use of NRTPs as reporter molecules for use with endogenous or native inducers that target gene promoters within viable cells. The NRTPs of the invention can be engineered using the methods described in Section III and below in Examples 1-2.

In some embodiments, the method comprises employing a NRTP as a reporter, wherein the NRTP comprises a reporter gene that is operably linked to an inducible promoter that controls the expression of a target gene within a target cell. When the NRTP that includes the reporter gene is introduced into the target cell, expression of the reporter gene is possible via induction of the target gene promoter in the reporter nucleic acid molecule.

Transcripts

As described above, a transcript is a length of nucleotide sequence (DNA or RNA) transcribed from a DNA or RNA template sequence or gene. The transcript can be a cDNA sequence transcribed from an RNA template or an mRNA sequence transcribed from a DNA template. The transcript can be transcribed from an engineered nucleic acid construct. The transcript can have regions of complementarity within itself, such that the transcript includes two regions that can form an intra-molecular duplex. One region can be referred to as a “cis-repressing sequence” that binds to and blocks translation of a reporter sequence. A second region of the transcript is called a “reporter sequence” that encodes a reporter molecule, such as a detectable or selectable marker.

The transcripts of the invention can be a transcript sequence that can be 5, 6, 7, 8, 9, 10, 11, 12, 13, 14, 15, 16, 17, 18, 19, 20, 21, 22, 23, 24, or 25 nucleotides in length. In other embodiments, the transcript can be at least 25, 30, 40, 50, 60, 70, 80, 90, 100, 500, 1000, 1500, 2000, 3000, 4000, 5000 or more nucleotides in length. The cis-repressing sequence and the reporter sequence can be the same length or of different lengths.

In some embodiments, the cis-repressing sequence is separated from the reporter sequence by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, or more spacer nucleotides.

Vectors

In another aspect, the transcripts (including antisense and sense sequences) of the invention are expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). These sequences can be introduced as a linear construct, a circular plasmid, or a viral vector, including bacteriophage-based vectors, which can be incorporated and inherited as a transgene integrated into the host genome. The transcript can also be constructed to permit it to be inherited as an extrachromosomal plasmid (Gassmann, et al., Proc. Natl. Acad. Sci. USA (1995) 92:1292). The transcript sequences can be transcribed by a promoter located on the expression plasmid. In one embodiment, the cis-repressing and reporter sequences are expressed as an inverted repeat joined by a linker polynucleotide sequence such that the transcript has a stem and loop structure.

Recombinant expression vectors can be used to express the transcripts of the invention. Recombinant expression vectors are generally DNA plasmids or viral vectors. Viral vectors expressing the transcripts can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Eglitis, et al., Science (1985) 230:1395-1398; Danos and Mulligan, Proc. Natl. Acad. Sci. USA (1998) 85:6460-6464; Wilson et al., 1988, Proc. Natl. Acad. Sci. USA 85:3014-3018; Armentano et al., 1990, Proc. Natl. Acad. Sci. USA 87:61416145; Huber et al., 1991, Proc. Natl. Acad. Sci. USA 88:8039-8043; Ferry et al., 1991, Proc. Natl. Acad. Sci. USA 88:8377-8381; Chowdhury et al., 1991, Science 254:1802-1805; van Beusechem. et al., 1992, Proc. Natl. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

Any viral vector capable of accepting the coding sequences for the transcript(s) to be expressed can be used, for example, vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors featured in the invention can be pseudotyped with surface proteins from vesicular stomatitis virus (VSV), rabies, Ebola, Mokola, and the like. AAV vectors featured in the invention can be made to target different cells by engineering the vectors to express different capsid protein serotypes. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the transcripts into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference. Viral vectors can be derived from AV and AAV. A suitable AV vector for expressing the transcripts featured in the invention, a method for constructing the recombinant AV vector, and a method for delivering the vector into target cells, are described in Xia H et al. (2002), Nat. Biotech. 20: 1006-1010. Suitable AAV vectors for expressing the transcripts featured in the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosures of which are herein incorporated by reference.

The promoter driving transcript expression in either a DNA plasmid or viral vector featured in the invention may be a eukaryotic RNA polymerase I (e.g., ribosomal RNA promoter), RNA polymerase II (e.g., CMV early promoter or actin promoter or U1 snRNA promoter) or generally RNA polymerase III promoter (e.g., U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini et al., 1986, Proc. Natl. Acad. Sci. USA 83:2511-2515)).

In addition, expression of the transcript can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D-1-thiogalactopyranoside (IPTG). A person skilled in the art would be able to choose the appropriate regulatory/promoter sequence based on the intended use of the dsRNA transgene.

Generally, recombinant vectors capable of expressing transcript molecules are delivered as described below, and persist in target cells. Alternatively, viral vectors can be used that provide for transient expression of transcript molecules. Such vectors can be repeatedly administered as necessary. Once expressed, the transcript binds to target RNA and modulates its function or expression. Delivery of transcript expressing vectors can be systemic, such as by intravenous or intramuscular administration, by administration to target cells ex-planted from the patient followed by reintroduction into the patient, or by any other means that allows for introduction into a desired target cell.

Transcript expression DNA plasmids are typically transfected into target cells as a complex with cationic lipid carriers (e.g., Oligofectamine) or non-cationic lipid-based carriers (e.g., Transit-TKO™). Multiple lipid transfections for dsRNA-mediated knockdowns targeting different regions of a single PROC gene or multiple PROC genes over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

The delivery of the vector containing the recombinant DNA can by performed by abiologic or biologic systems. Including but not limited to electroporation (as described in the Examples), liposomes, virus-like particles, transduction particles derived from phage or viruses, and conjugation.

Reporters for Transcript Assay

In some embodiments, the nucleic acid construct comprises a reporter sequence (e.g., a reporter gene sequence). The reporter gene encodes a reporter molecule that produces a signal when expressed in a cell. In some embodiments, the reporter molecule can be a detectable or selectable marker. In certain embodiments, the reporter molecule can be a fluorescent reporter molecule, such as a green fluorescent protein (GFP), yellow fluorescent protein (YFP), cyan fluorescent protein (CFP), blue fluorescent protein (BFP), or red fluorescent protein (RFP). In other embodiments, the reporter molecule can be a chemiluminescent protein.

Reporter molecules can be a bacterial luciferase, an eukaryotic luciferase, a fluorescent protein, an enzyme suitable for colorimetric detection, a protein suitable for immunodetection, a peptide suitable for immunodetection or a nucleic acid that function as an aptamer or that exhibits enzymatic activity.

Selectable markers can also be used as a reporter. The selectable marker can be an antibiotic resistance gene, for example.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Identification of De Novo Bacteriophages from A. baumannii Isolates and Production of Non-Replicative Transduction Particles (NRTPs)

Materials and Methods

Bacterial Strains and Growth Conditions

A set of 288 A. baumannii clinical isolates were collected from the CDC, Pasteur Institute, and IHMA, Inc., and species-verified in house by biochemical testing and MALDI-TOF. Each A. baumannii-verified isolate was identified with Abi′ followed by a number based on order of accessioning. Isolates were cultured in Luria-Bertani (LB) broth at 37° C. with 225 rpm agitation, or on LB agar plates at 37° C. in stationary conditions. Strains harboring the packaging plasmids were selected for by growth in LB Lennox (low-salt) broth or agar supplemented with either 150 μg/ml or 250 μg/ml hygromycin B Gold (Invivogen, San Diego, Calif.) in E. coli cloning strains or A. baumannii isolates, respectively. A. baumannii strains with gene-disrupted terminase regions were selected for in LB Lennox (low-salt) broth or agar supplemented with 200 μg/ml zeocin (Thermo Fisher Scientific, Carlsbad, Calif.) or phleomycin (Invivogen, San Diego, Calif.).

Induction and Purification of Lysogenic Phage

A. baumannii lysogenic strains were grown to log-phase (OD₆₀₀˜0.6-0.8) in LB broth at 37° C. with a shaking speed of 225 rpm before the addition of 4 μg/ml mitomycin C to induce any lysogenic phage that may be present. After the treatment of mitomycin C for 30 minutes, the cells were centrifuged for 10 min at 3750 rpm, and resuspended in fresh LB broth. The cells were incubated at 37° C. with a reduced shaking speed of 150 rpm for 4 hours. Phage-containing supernatant was centrifuged to pellet the cellular debris for 10 min at 3750 rpm, and passed through a 0.2 μM filter unit to remove any remaining cellular debris. Lysates were stored in the dark at 4° C. until use.

Host Range Evaluation by Plaquing Method

Each of the 288 A. baumannii clinical isolates was grown in LB broth supplemented with 5 mM CaCl₂). Upon reaching log phase at OD₆₀₀˜0.4-0.6, 300 μl of the bacterial culture was added to 4 ml of melted top agar consisting of 0.5% agar and 5 mM CaCl₂). The top agar and bacterial culture mixture was poured over a plain LB agar plate, and immediately spotted with 5 μl of filtered lysate. The plates were incubated overnight at 37° C., and the following day scored for the presence or absence of defined plaques.

Phage Genomic DNA Sequencing of Inducible De Novo Phages

Phage genomic DNA was isolated from de novo Abi phi 33, 49, and 147 phages yielding broad plaquing host range. Lysates were centrifuged at 14,000 rpm for 2 hours to pellet the phage, and resuspended in 1×SM buffer. Phage lysate was treated with DNase I (Thermo Fisher, Carlsbad, Calif.) and processed with a phage DNA isolation kit (Norgen Biotek Corp., Thorold, ON, Canada) to isolate phage genomic DNA (gDNA). Purified phage gDNA samples were sent to ACGT, Inc. (Wheeling, Ill.) for de novo phage genome sequencing, assembly, and putative annotation of open reading frames using the MiSeq sequencing platform (Illumina, San Diego, Calif.).

Visualization of Phages by Transmission Electron Microscopy

To pellet the phages, phage lysates were centrifuged at 14,000 rpm, room temperature for 2 hrs, and resuspended in 50-100 μl SM buffer. Samples were submitted to University of Colorado, Boulder Electron Microscopy Services (Boulder, Colo., USA) for negative staining and transmission electron microscopy.

Construction of A. baumannii Plasmids for Phage Packaging

Plasmid construction is detailed in the Results section. All plasmids used in this study are listed in Table 1.

TABLE 1

| Plasmid | Origins of | | luxAB | | TT | Antibiotic |
|---|---|---|---|---|---|---|
| accession | replication (E. coli; | luxAB | (WT or | terSL | flanking 3′ | selection |
| number | A. baumannii) | promoter | mutant) | (source; length) | end of luxAB | marker(s) |
| p3028 | pUC; pWH1266 | pBla | WT | none | no | kanR; zeoR |
| p3033 | pUC; pWH1266 | pBla | WT | Abi 147; terSL | no | kanR; zeoR |
| p3066 | pUC; pWH1266 | pBla | WT | Abi 147; 250 | no | kanR; zeoR |
| | | | | bp upstream | | |
| | | | | of terS + terSL | | |
| p3073 | pUC; pWH1266 | pBla | WT | Abi 147; 250 | yes | hph |
| | | | | bp upstream | | |
| | | | | of terS + terSL | | |
| p3074 | pUC; pWH1266 | pBla | mutant | Abi 33; 250 bp | yes | hph |
| | | | | upstream of | | |
| | | | | terS + terSL | | |
| p3075 | pUC; pWH1266 | pBla | mutant | Abi 49; 100 bp | yes | hph |
| | | | | upstream of | | |
| | | | | terS + terSL | | |

Design and Construction of Terminase Region Deletion

The gene disruption method developed by Aranda, et al. (Aranda, et al., 2010) was used to replace the terminase region with a selection marker via double-crossover recombination. The substrate for recombination consisted of the zeocin resistance cassette (zeoR) flanked at the 5′ end with 600 bp of the terS upstream region, and at the 3′ end with 600 bp downstream of terL. Each of these sequences were designed specifically for Abi 33, Abi 49, and Abi 147 terminase regions, synthesized as gBlocks (IDT, Redwood City, Calif.), subcloned into pCR-BluntII-TOPO vector, and PCR amplified with Phusion High-Fidelity DNA polymerase (New England Biolabs, Ipswich, Mass.). The linear, recombinant DNA was purified using a PCR purification kit (New England Biolabs) and concentrated to achieve a concentration of ˜5 μg/ml.

Preparation of Electrocompetent Cells for Generating Terminase Region Knock-Outs

A. baumannii strains were made electrocompetent using a method adapted from Jacobs, et al. (Jacobs A C, et al., 2014). Bacterial cultures were grown overnight in 50 ml of LB Lennox broth at 37° C. with shaking at 225 rpm. The cultures were transferred to 50-ml conical tubes and centrifuged for 10 min at 3750 rpm, room temperature, to pellet the cells. All subsequent steps were performed at room temperature. The supernatant was removed by pipetting, and the cell pellet was gently resuspended in 25 ml (half the starting culture volume) with 10% (v/v) glycerol. The cells were pelleted for 10 min at 3750 rpm, and the wash with 10% glycerol was repeated. Pelleted cells were resuspended in 1.5 ml of 10% glycerol.

Transformation of Gene Knockout Products Via Electroporation

The linear, recombinant DNA (≤10 μl) was mixed with a 50 μl aliquot of fresh electrocompetent cells. The mixture was placed in a 1-mm electroporation cuvette (Bulldog) and pulsed in a Bio-Rad Gene Pulser at 25 μF, 100 ohm, and 1.8 kV. Cells were incubated in 900 μl of SOC broth (Invitrogen) at 37° C. for 2-3 hr to allow recovery of the cells and recombination events to occur. Cells were plated on LB Lennox agar plates containing 200 μg/ml of zeocin or its derivative, phleomycin.

Transformation of Terminase Knock-Out Strains with Complementing Plasmids

Terminase knock-out strains were grown overnight in LB Lennox containing 200 μg/ml of zeocin or its derivative, phleomycin. Cultures of Abi 33, Abi 49, and Abi 147 terminase knock-out strains were made electrocompetent as described previously, and transformed with plasmids p3074, p3075, and p3073, respectively. Transformants were selected on LB Lennox agar containing 200 μg/ml of zeocin and 250 μg/ml of hygromycin B Gold.

Induction of Mutant Strains to Create Lysate of Non-Replicative Transduction Particles

From freshly streaked colonies, Abi 33 ΔterSL::p3074, Abi 49 ΔterSL::p3075, and Abi 147 ΔterSL::p3073 were grown overnight in LB Lennox broth containing 250 μg/ml of hygromycin B Gold at 37° C. with 225 rpm agitation. Cells were inoculated with 3% overnight culture into LB broth and incubated at 37° C. with 225 rpm agitation until OD600 reached 1.6-1.8. To induce phage production, 4 μg/ml of mitomycin C (Millipore Sigma, St. Louis, Mo.) was added to the culture for 40 min at 37° C. with 150 rpm agitation. To remove the mitomycin C, cells were centrifuged at 3750 rpm for 10 min at room temperature and the pellet was resuspended in fresh LB broth. The culture was incubated overnight at 37° C. with 150 rpm agitation. To remove cellular debris and purify each of the lysates, cultures were centrifuged at 5000 rpm for 15 min. The non-replicative transduction particle lysate-containing supernatant was passed through 0.2 μm Thermo Scientific™ Nalgene Rapid Flow filters (Thermo Fisher) and stored protected from light at 4° C. until use.

Concentration of Lysates Containing Non-Replicative Transduction Particles

Crude lysate was centrifuged at 10,000×g at 4° C. for 15 minutes to remove cell debris and filter sterilized by passing through 0.2 μm Thermo Scientific™ Nalgene Rapid Flow filters. The sterile lysate was centrifuged at 30,000×g at 4° C. for 16-18 hours to pellet the transduction particles. After removing the supernatant, the phage pellet was resuspended in 1×SM buffer (100 mM NaCl, 8 mM MgSO4, 50 mM Tris-HCl) to 10×- or 20×-fold concentration, and filter sterilized again through 0.2 μm Thermo Scientific™ Nalgene Rapid Flow filters.

Detection of Luminescence in Target Strains with Non-Replicative Transduction Particles

A glycerol stock of 96-well strain panel was inoculated into 500 μl of LB Miller broth in a deep-well plate for overnight growth at 37° C. with 500 rpm agitation. Day cultures were prepared by inoculating 1 μl of overnight cultures in 800 μl LB Miller broth supplemented with 25 mM CaCl₂) and 50 mM MgCl₂ for an approximate 107 cfu/ml starting cell load. Cells were grown at 37° C. with 500 rpm agitation for 1.5 hr. In a white 96-well plate, 120 μl of the day culture and 50 μl of transduction particles were mixed. For the transduction particles cocktail assay, 120 μl of the day culture was mixed with 25 μl of each 3×-concentrated Abi 33, Abi 49, and Abi 147 transduction particles. The assay plates were incubated for 2 hrs at 37° C. with 100 rpm agitation, followed by a cooling step for 30 min at 30° C. with 100 rpm agitation to allow for optimal luxAB expression. Plates were read on a SpectraMax L instrument (Molecular Devices, San Jose, Calif.) with nonanal as substrate for relative light units (RLU) emission.

Host Range Assessment by Transduction Particles (Tc) Spotting

The host ranges of Abi 33, Abi 49, and Abi 147 transduction particles were assessed by spotting 5 μl of cells incubated with (see Westwater paper). Immediately prior to luminescence assay readings, cells were spotted onto LB Lennox agar plates containing 250 μg/ml hygromycin B. Plates were incubated at 37° C. overnight and scored the next day for the presence or absence of bacterial growth (i.e., transductants harboring the plasmid conferring hygromycin resistance). To confirm the transductants and remove any naturally hygromycin-resistant cells, the colonies were resuspended in LB broth and tested for RLU emission.

Results

Inducible, Lysogenic A. baumannii Phages with Broad Plaquing Host Ranges were Identified and Characterized

To identify and narrow down the inducible and broad host range prophage candidates, a collection of 288 unique A. baumannii clinical isolates was accessioned and individually treated with mitomycin C, a potent inducer of the bacterial SOS response, to induce any lysogens harbored by the bacteria to convert to the lytic cycle. The lysates from this preparation were spotted onto a subset of the same A. baumannii strains using a phage plaquing method. The presence of plaques was scored and maintained in a spreadsheet for host range. Cumulatively, lysates with positive plaques were ranked based on host range and complementary coverage. The lysates with broadest host range were identified as Abi 33, Abi 49, and Abi 147 with plaquing host ranges of 25% (72/287), 23% (61/269), and 4% (7/163), respectively, and 32% (93/288) cumulatively.

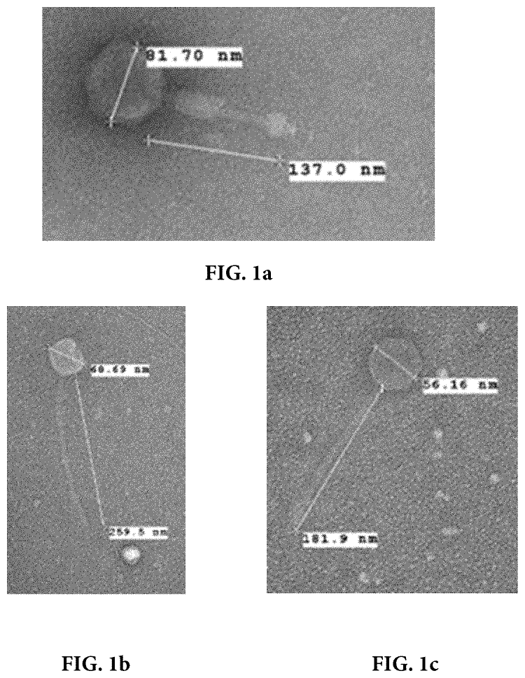

The phage genomic DNA from the Abi 33, Abi 49, and Abi 147 lysates was purified and sequenced to identify the phage packaging (i.e., terminase) regions. The complete phage genomes sizes for Abi 33, Abi 49, and Abi 147 were 53, 40, and 36 kb, respectively. Abi 33, Abi 49, and Abi 147 phages were imaged by transmission electron microscopy to confirm the presence of intact phages, determine the homogeneity of the phage population, and identify phage family based on morphology. Abi 33 lysate contained a heterogenous population of Myoviridae phages; Abi 49 and Abi 147 lysates contained a homogenous population of Siphoviridae phages (FIG. 1a, 1b, 1c).

Non-replicative transduction particles can be created by deletion of the host strain terminase region and complementation on a phage packaging plasmid

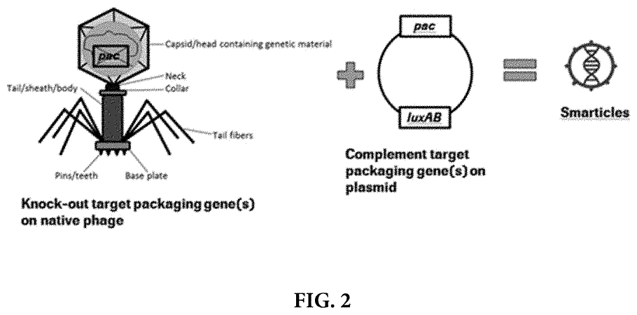

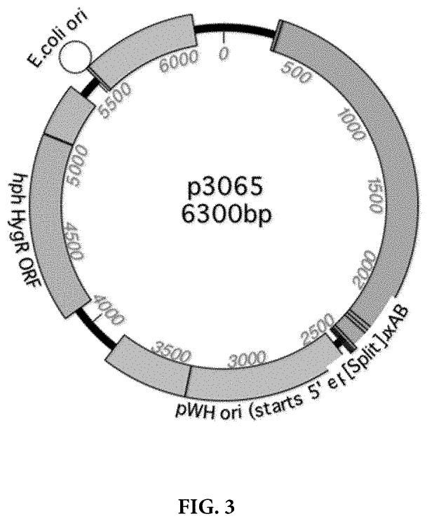

Non-replicative transduction particles technology relies on deletion and complementary to generate engineered prophages carrying the reporter DNA instead of their native phage DNA. Abi 33, Abi 49, and Abi 147 transduction particles were synthesized in which each consisted of packaging-deficient phage shells harboring plasmids with namely, 1) the phage packaging genetic elements to complement the loss on the host phage genome, and 2) the reporter genes, luxAB, for the luminescence assay (FIG. 2). On the host Abi 33, Abi 49, and Abi 147 genomes, the terminase subunits encoded by terSL as well as ≤250 bp upstream region were knocked out for each strain and replaced with a zeoR selection cassette. The backbones of plasmids were constructed for phage packaging to complement the loss of the terminase region on the Abi 33, Abi 49, and Abi 147 host strains (FIG. 3). The actual nucleotide sequences in the terminase regions that were deleted are shown in Table 2

TABLE 2

| | SEQ | |
|---|---|---|
| Description | ID NO: | Sequence |
| Abi 33 terSL | 1 | ATGGCTGCACTTAAAGAACAGGTAAAAATATTTATTGTTC |
| region | | AAGCGCTTGCCTGCATGGATACCCCTCAACAGGTAGCTAA |
| deleted: terS | | TGCTGTCAAGCAAGAATTTAACATTGAGATTGATCGAAAA |
| (full), terL | | CAGGTACAACTTTATGACCCGACAAAAGCGGCAGGAAAGA |
| until 150 bp | | ATTTAAGTAAGAAATATAAAGACCTTTTTCATAAAACCCG |
| before 3′ | | AGAGGACTTTAAAAAGAATGTTTATGACATCCCTTTAGCT |
| stop codon | | AATAAAGCCTATCGGCTTAAAGAACTTCAGAAGATCTATG |
| | | AAGACTGGAAGAACAACAGGCTTATGAAGCAAGGGGTTAT |
| | | TAAACAGGTTAGGGAAGAAATGCAGGGTTATGACCTCATG |
| | | CTTTTAAATCTTGAGTTAAAGCAACTTGAGATTGAAAAGT |
| | | TAAGAGAGGGTGAAGGTGATGAAGATCCAACACCAGTCAA |
| | | GGTAACTATTCAAGTTGTAGATGCGAGTAAAAAAGATGCC |
| | | GAACATCAATCCGACACTGAATGTACCTCAGGCTAATTTT |
| | | TTGCAGATGGAAAAGAAGTTCCGCGCATTTGTCGCTGGCT |
| | | TTGGATCGGGAAAGACTTGGGTTGGATGCTCCAGTTTATG |
| | | CAACAAAGCTTGGGAATTCCCAAAAGTACCTTTGGGTTAT |
| | | TTTGCTCCAACTTACCCGCAGATTCGCGACATTTTCTTTC |
| | | CAACTATTGAAGAGGTTGCTTTCGATTGGGGGCTTAAAAC |
| | | TAAGGTTTATGAAACCAATAAAGAGGTGGATATCTATTAT |
| | | GGTCGGCAATATCGAACTACAATCATTTGCCGGTCTATGG |
| | | AGAAACCAGCAACCATTGTAGGTTTTAAAATTGGCCACGC |
| | | CTTGATTGATGAGCTTGATGTTATGGCCAAGGTCAAAGCT |
| | | CAACAGGCTTGGCGTAAGATCATCGCTCGTATGCGTTATA |
| | | AGCAAGCTGGTTTGCTCAACGGTATTGATGTGGCCACAAC |
| | | ACCAGAAGGTTTTAAGTTTACATACGAGCAATTTGTTAAA |
| | | GAGGCAAATAAATCAGAGGCTAAGCGTAAGCTCTATGGAA |
| | | TGATTCAAGCTTCAACTTATGACAATGAAGCTAATCTTCC |
| | | AGATGACTACATATCATCACTTTATGAGTCTTATCCGCCG |
| | | CAATTAATTTCAGCTTATTTAAGAGGGCAGTTTGTCAATT |
| | | TAACCAGTGGTGCTGTTTACCCCGACTTTGATCGAGTTCT |
| | | AAACCACACGGATGAAGAAATTAAGAAAGGTGAGCCTTTA |
| | | CTCATTGGTATGGATTTTAACGTGCTTAAAATGGCTGCTG |
| | | TGGTTTATGTCATTAGAGAAGGGAAGCCAAGAGCTTTAGA |
| | | TGAACTGGTTGGCGTGAGAGATACACCGACGATGTGTCAA |
| | | CTGATTAATGAGCGCTTTCCAGATCACGATATTACTGTGA |
| | | TTCCAGATGCTTCAGGTCAGGCAACATCATCAAAGAACTT |
| | | CAGTGAATCTGATCATGCAATCTTAAAGAAAAATGGATTC |
| | | AAAGTTGAAGTTAATGGTGTGAATCCCGGTATTAAAGATC |
| | | GTATCACTGCAGTTAATGCACAAATTCTGAATGCTGAGGG |
| | | TGAACGACACTTAAAAGTGAACACAAACAAGTGTCCTAAC |
| | | TTTACGGCTACTTTAGAA |
| Abi 49 terSL | 2 | GCCTAATGGCTCTTTTTTTTGCCCATTTTGTTATACGTAG |
| region | | TTATACGATGAGGAAGTTATGGCGACACTAAAAGAGCCTG |
| deleted: 58 | | TGAAAATCTTTATAGTTCAGTCTCTTGCTTGTCGTGATAC |
| bp upstream | | ACCTCAAGAAGTGGCTGAACTCGTAAAACAAGAATTTGGC |
| of terS, terS | | GTTGATATAGATCGTGTTCAAGTTGCAACATATGACCCTA |
| (full), and | | CAAAAGTTGCTGGTAAGAACTTAAGCAAAAAGTATGTCGA |
| terL until 92 | | ACTATTTGAAAAAACCAGAGATGAGTTTGATAAAGGCTTA |
| bp before 3′ | | ATTGATATTCCTATTGCTAATAAGTACTACCGATTGAAGC |
| stop codon | | AATACCAAAGACAACTTGAGAAGACTAGAAACGTTAAAAC |
| | | AGCCTTAAAAATTCTTGAGCAAGCCGCTAAAGACATTGGT |
| | | GGTCAATTTACTAATCGCCAAGAAATTACAGGCAAAGACG |
| | | GCGGACCAGTCCAAACAGTTAATTCTGAAATTCAAGTTCC |
| | | AATGGAAGATTACTTAAAAGCACGGAGGGAAGTCTTAGAT |
| | | GAGTACTGATGCGGCTCGGGATAAAGCCATCCGGATCGAG |
| | | GCGCAAGAAGATTTATATTTCTTCACAAGGTACATGTTTA |
| | | AGGAGCGCCGTGGTTATAAATGGATGCAAAATTGGCACCA |
| | | CTTAGAAATCTGCGAAGCTTTAATGAAAGTTTATCGCGGA |
| | | GAGATAAAGCGGTTAATTATTAACGTTCCACCACGATATT |
| | | CTAAAACTGAAATTGCTGTAATTAATTTCATGGCTTGGTG |
| | | TTTTGGTAAGAATCCAGACTGTGAGTTTATTCATATCAGT |
| | | TACTCGGCAATGCTTGCCGCAAATAATGCCTTCCAGATTC |
| | | GAACACTCGTACAAGAGGAGGCGTATAAAAAGGTCTTTCC |
| | | TGATCTTACATTGCGTGATGATAGTAAGGCTAAAGACTTC |
| | | TGGAGAACTTCTCAAGGCGGTGTCTGCTATGCGACTGGTA |
| | | CAGGCGGTACGATTACTGGTTTTGGCGCAGGTAAACTTCG |
| | | TGATGGGTTTGGTGGATGCATCATTATCGATGACCCACAC |
| | | AAAGCGCATGAAGCTTCTTCTAAAACAATTCGAGAAGGGG |
| | | TAATTGATTGGTTCCAAAACACCCTTGAGTCGCGTACTAA |
| | | CTCACCAGATACGCCGATCATTGTGATTATGCAGCGACTT |
| | | CATGAAGATGATTTGGCTGGTTGGTTGTTAGGCGATAGAA |
| | | AAGATGGCGTTCCTGTAGCTGGTGGTAACGGTGAGGTATG |
| | | GGAACATCTTTGTCTTTCTGCTATTCAGGAAGACGGATCC |
| | | GCACTATGGCCAGCAAAACACAATATCCAAAAGTTAAGGC |
| | | AAATGGAGCAAGCTGCGCCGTATGTATTTGCAGGGCAATA |
| | | CCGACAAATGCCATCACCGCCAGCAGGCGGTTTTTTTAAG |
| | | CCTGACAATATTCAAATTGTTGATGCTTTGCCTGCGGATG |
| | | TTTTGAAACAAGTGAGGGCTTGGGACTTCGGAGCGACCGA |
| | | AAACGAAGGCGACTTTACTGTAGGTGTAAGAGAAGCTCTA |
| | | GGTGCAGATGGTTTTACTTACATTGTCGATGTTACTAGAG |
| | | GACAGCTTGGTCCAGACAATGTGAATAAGCGCTTAGAACA |
| | | AACCGCAAAAATAGATGGGAAAAAAGTTTCTGTGCGTCTA |
| | | CCACAAGATCCCGGTCAAGCTGGTAAATCGCAAGCTAGTT |
| | | CATTTGTGAAGCTTCTTGCGGGTTATAGCGTGATAGCTAA |
| | | GCCAATTTCAGGTGACAAGCTTACACGTGCACAACCATTT |
| | | GCGGCCCAAGTTAACGTAGGAAATGTACGAATGCTCAAAG |
| | | GTGAATGGAATAAGGATTTTATTGATGAGCTTCGTCATTT |
| | | TC |
| Abi 147 | 3 | GGATGTCAAAACTTCCAAGCCCTTCGCCGTTGGACACCGC |
| terSL region | | CCCCCATCGCACGCACAAAAAAAATTCCCTCTCAGAAAAA |
| deleted: | | GTTAAAGCAAAAAGTTAAAATCAAGTTAAAGGTAGAGCAA |
| 128 bp | | TGGCATTAACAGAGAAAATGGAAAAATTTGCTCTTGCCAT |
| upstream of | | TGTTGACGGCAAGACAAATAAAGAAGCAGCAATTTCAGCA |
| terS, terS | | GGTTATGCGGAAAAAACTGCATCCGCCGCAGGTGCTAGGT |
| (full), terL | | TAGCAAAAGATCCTGAAATTATTGTCTATATTGAAATGTT |
| (full) | | AAAGGCCCAAAAAGAAGGGCGCTCTTTAACATCTAATTCA |
| | | CCAAAAGTTAAACCTAAAGATACACCCGAAAATAGTGGTG |
| | | AAGATGAAAACCCTATTGAGGAATTTCAATTTGAAGGTGA |
| | | TGATCCTTTAGATTTTTTAATTAAGGTCATGAACTTCAAT |
| | | GGCAACAAGCTGCCACTTAGAATGCAAGCAGCAATTGCAG |
| | | CATTGCCTTATAAACACGGGAAGGTTGCGGAAAAAGGCAA |
| | | AAAAGAAACTAAACAAGACAAGGCAAAGGAAGCGACCAGA |
| | | ACAGGAAAATACGCCACATTGGACAATCAGTTGCCAAGCT |
| | | AACTATTAAGAGGAACTTTGCATGCAAAGTCTAGAATACG |
| | | AAACCGTAAGTGGTGAAACTATCACTATTCAAAATATCAA |
| | | AGATGGCCCTTGCTGTCATGACTCTATTGAAAAGTTGCCA |
| | | GCAACAGAAAGGTTGGTGAAGATTACTTATCAATGTCGCA |
| | | AGTGCTTTTCCAGATTTTCCGAAGAAGATTATCAATTGAT |
| | | TGTTAATCAATAAAAGGTTTTGTATGGATCCGTAGGCGAT |
| | | ACGGTGCGTTGGAGGAAGGAGGACCACAACTGCCAACGTA |
| | | ATAACCCGCTAGCAGTGGGCGAAACAGCGTAGTTAAAGCA |
| | | GGGGTTCGCAACCTGTCATACAAATTTATTCCGCCTTCGG |
| | | GCGGTTTTTTCATGGACCATTTAAATGACTGCAAAACTAC |
| | | CAGACTGGACTACAGCTTGCCCAGACTGGGCGACCCGTAT |
| | | TGTTTCTAAACAATCGTTAATGCCGTGTAAGCCATTATTC |
| | | CCCAAAGTGGCTGACGTAGCGGAGCGTATCTTTAAAGAGT |
| | | TAATTCTTGTTGATGTGATGGGTAGCCCTAAGATGGGTGA |
| | | TGTCACATTGGAATGGGTGATCGAGTTTGTTCGTGCAATC |
| | | TTTGGCGCATATGATCCAAGCACAAAGCGCAGATTAATTC |
| | | GTGAATTCTTTCTTTTGATTTCGAAGAAGAATACTAAATC |
| | | TACGATTGCCGCCGGCATTATGCTTACTGCATTAATTCTT |
| | | AATGATCGACAATCTGCCGAACTAATTATTCTTGCGCCTA |
| | | CTAAAGAAGTTGCTGATAACTCATTTAATCCAATCCGGGA |
| | | TTTCATACGCGCAGATGAAGAATTAAGTGAAAGATTTAAT |
| | | GTATCTGAGCACACAAAAACAGTTACGCATCTAGGTACCG |
| | | GAGCAACACTTAAAGTTATTGCAGCAGAATCTAACGCTGC |
| | | AGCTGGTAAGAAAGCTTCAATCATTTTGATAGATGAGGTC |
| | | TGGCTATTCGGGAAACGTGCCAACGCTGAATCAATGTTCC |
| | | GTGAAGCAAAGGGTGGTTTAGCATCTCGTCCAGAAGGTTG |
| | | TGTGATTTATCTGTCTACCATGTCGGATGAAGTGCCATGT |
| | | GGAGTATTTAAGCAGCTTTTAGATTATGCCAGAGATGTAC |
| | | GTGACGGAATTAAAGTTGATAAAAGTTTTCTACCACTTAT |
| | | TTATGAATTCCCTAAGCATCTTGTAGAAGCAGGCGAACAT |
| | | TTAAAACCTGAAAATTTCTACATCACAAACCCAAACTTGG |
| | | GTGCTTCGGTTGATCTTGAATATCTGATTTCGGAATTTAA |
| | | CAAAGTTAAAGATGCTAGTGAAGAATCTCTTAGAGACTTC |
| | | TTGGCCAAACACTTAAACATTGAAATCGGCATGAACCTTC |
| | | GTGCTAACCGGTGGGCGGGTGCAGAGTATTGGAATGCTCA |
| | | AGCTAAAGATATCCAAATCGACCAACTAATTGAGCTATCC |
| | | GATGTCATTACTTTGGGTATTGATGGCGGTGGTCTCGACG |
| | | ACTTACTTGGCTTCGCTGCTTTAGGTCGTTTAACAGAAGA |
| | | TCCTCGTATCTGGTGGCTATGGAATCATGCATGGGCAAAT |
| | | AAGATTGCTTTAGAGCGCAGAAAAGAGAATGTGCCTAAGT |
| | | ATGAAGACTTCAAGTCTGAGGGTTCTCTAACTGTTGTTGA |
| | | CCGAATAGGCGATGACATTGACCAACTCGCAGCAATTGCT |
| | | AAGAAGGTTTATGACAGTGGAAAGCTTAATAAGATCGGAC |
| | | TAGATCCATTGGGCTTAGGCGGTCTTTTAGATGGCTTACT |
| | | TGAGGCAGGAATTCCAGAGGAAAGCATGTTTGCTGTGCCA |
| | | CAAGGCTACAAACTCATGTCCTACATCCTTACTACTGAGC |
| | | GCAAATTGGCAGAAGGCAATCTGTACCATGCTGGACAACA |
| | | GCTAATGACTTGGGCGGCAGGTAATGCCCGTGTCGTGATG |
| | | GTCGGCAATGGTATGCGAATAACCAAGCAAGAATCAGGTG |
| | | TTGGGAAGATTGACCCATTGATTGCCACATTTAACGCAGT |
| | | TGCTTTGATGTCAAGCAATCCTGAGCCTGCCAATCGCGTT |
| | | GATATTGACGAATACTTAGAGGATGTCGTGATAGCATGA |

Each shuttle plasmid backbone contained two origins of replication for E. coli and A. baumannii, respectively: pUC18, derived from pCR-Blunt II-TOPO vector (Thermo Fisher Scientific, Carlsbad, Calif.), and pWH1277, derived from the pWH1266 plasmid isolated from Acinetobacter calcoaceticus (Hunger M, et al., 1990). The phage-packaging plasmids contained an hph cassette encoding for hygromycin B resistance, derived from pMQ300 plasmid (provided by Prof. Robert M. Q. Shanks, University of Pittsburgh, Pa.), for near-universal antibiotic selection in all clinical A. baumannii isolates. Phage-packaging plasmids p30′73, p30′74, and p3075 were generated by cloning in the upstream region of the terminase region and the full-length terminase subunits, terSL, of Abi 147, Abi 33, and Abi 49, respectively. Specifically, p3073 contained 250 base pairs upstream of the terS ORF and terSL from Abi 147; p3074 contained 250 base pairs upstream of terS ORF and terSL from Abi 33; and p3075 contained 150 base pairs upstream of terS ORF and terSL from Abi 49. Plasmids p3073 contained wild-type, pBla promoter-driven luxAB from Vibrio fischerii; p3074 and p3075 also contained pBla promoter-driven luxAB from V. fischerii with two point mutations, C170R and N264D, in LuxA for improved luciferase activity. Plasmids p3073, p3074, and p3075 had an rrnG transcription terminator (TT) inserted at the 3′ end of luxAB. The TT region was derived from the OXB19 plasmid (Oxford Genetics Ltd., Oxford, U.K.).

A cocktail of Abi 33, Abi 49, and Abi 147 Smarticles yields high inclusivity assay results on a panel of unique A. baumannii clinical isolates

Abi 33, Abi 49, and Abi 147 non-replicative transduction particles were generated by a phage induction method and 3×-concentrated by centrifugation. A panel of 96 unique A. baumannii clinical isolates was grown to ˜107 cfu/ml cell load prior to incubation with the Abi 33, Abi 49, and Abi 147 Smarticles for 2.5 hr. Upon injection with the luminescence reaction substrate, 47%, 54%, and 59% of the strains were RLU-positive by Abi 33, Abi 49, and Abi 147 individual transduction particles, respectively (Table 2). As a cocktail, there was an additive effect as 82% of the strains were RLU-positive (Table 2).

TABLE 3

| | Phi 147 | Phi 33 | Phi 49 | Phi 33/49/147 |
|---|---|---|---|---|
| | NRTP | NRTP | NRTP | NRTP Cocktail |
| Abi Strain | 59% | 47% | 54% | 82% |
| Panel | (56/95) | (45/95) | (51/95) | (78/95) |

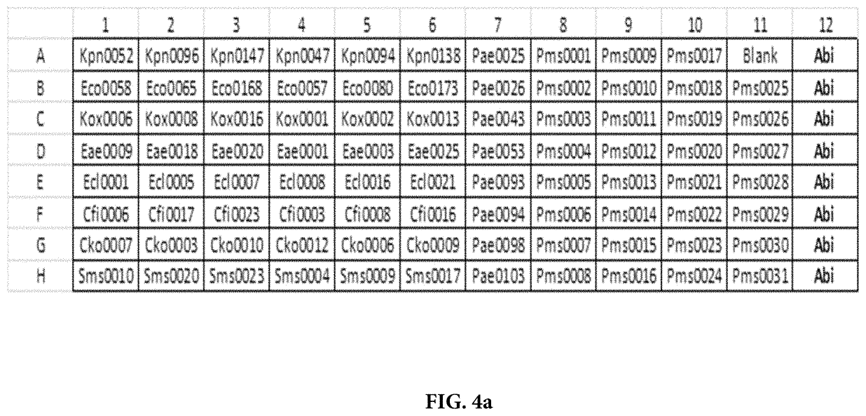

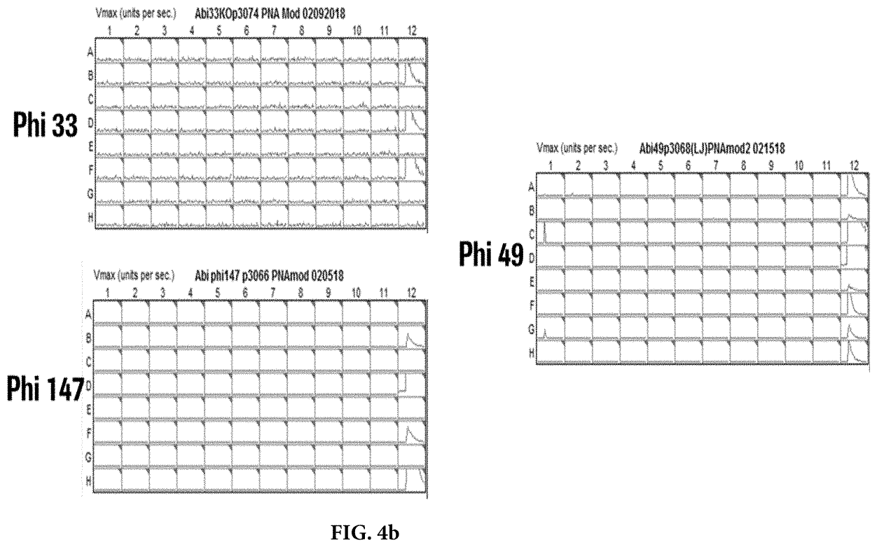

Abi 33, Abi 49, and Abi 147 transduction particles were generated by a phage induction method and tested for cross-reactivity on a panel of Gram-negative bacterial strains with a subset of A. baumannii strains to serve as positive controls in the assay (FIG. 4a). The panel of strains was grown to ˜107 cfu/ml cell load prior to incubation with the Abi 33, Abi 49, and Abi 147 Smarticles for 2.5 hr. No cross-reactivity was observed in the liquid assay against non-A. baumannii strains (FIG. 4b).

### Example 2: Stabilized Packaging Plasmids and Production of NRTPs

Construction of a new series of A. baumannii (Abi) packaging plasmids A stability issue was observed with lysogenic strain Abi33 Δ terSL::p3074, which lost the plasmid after a single passage. Similar plasmid instability issues were also observed in other Smarticles NRTP studies. It was hypothesized that this effect may have been due to leaky expression from the upstream hph promoter of the terminase gene, resulting in plasmid self-cleavage and plasmid loss from overactive terminase activity. For certain packaging plasmids, when the terminase was in the same orientation as hph, the strain was unstable and the Smarticles yielded poor coverage in the RLU assay. However, when the terminase orientation was flipped on the vector in the opposite direction to hph, then the strain stability and the Smarticles coverage improved significantly (data not shown). Thus, a new construct backbone was built by adding a transcriptional terminator after hph stop codon to improve 1) the strain stability, 2) possibly the packaging efficiency of plasmid into the phage head, and as a result, 3) Smarticles RLU assay coverage.

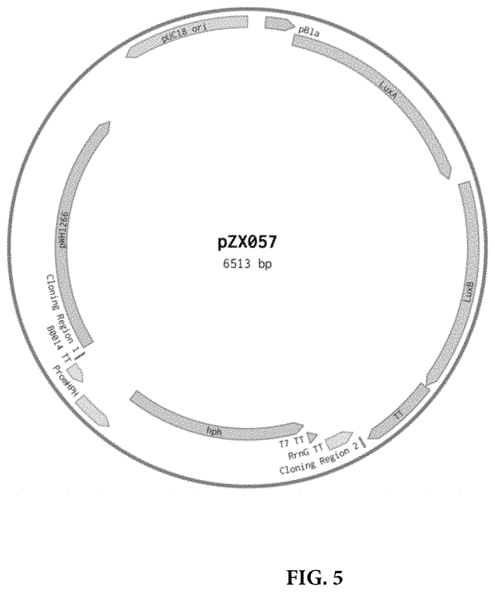

The empty A. baumannii packaging vector pZX057 (FIG. 5) was built with the following optimizations: 1) the luxA gene was replaced by the double-mutant luxA which is a more active enzyme and produces higher RLU signal in the assay; 2) bacterial transcriptional terminators T7 and rrnG were cloned after the hph gene to prevent the leaky expression of the downstream gene from the hph promoter; 3) the unutilized lacZ gene (residual from the original vector) was removed and replaced with another bi-directional double transcriptional terminator BBa-B0014 to prevent any unexpected leaky expression. Two cloning sites on the vector were used to clone in terminase gene regions as shown in the vector map.

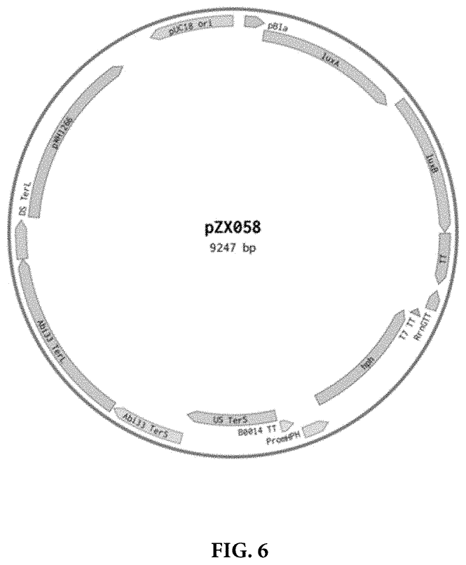

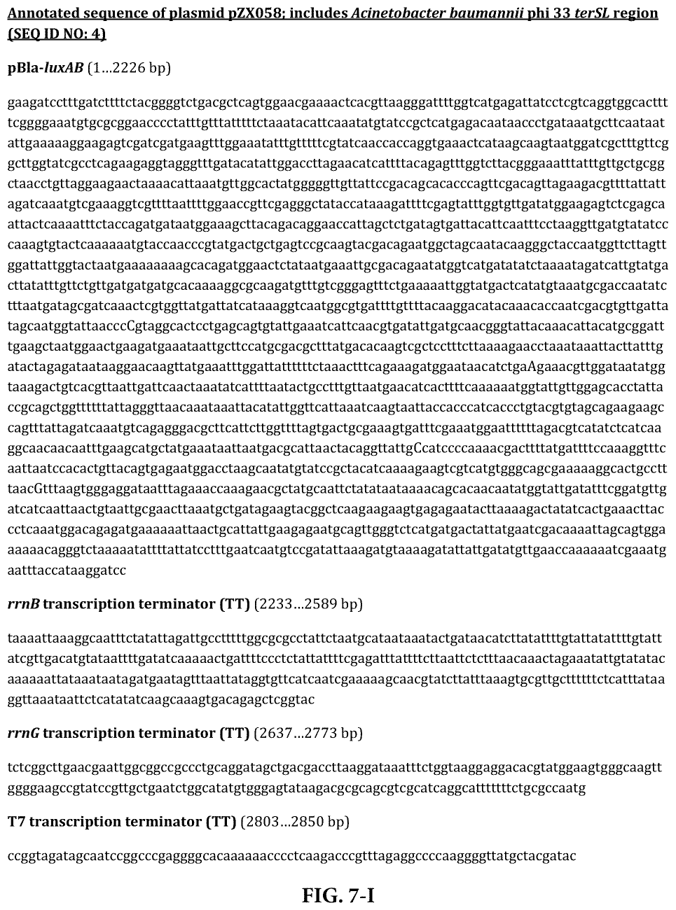

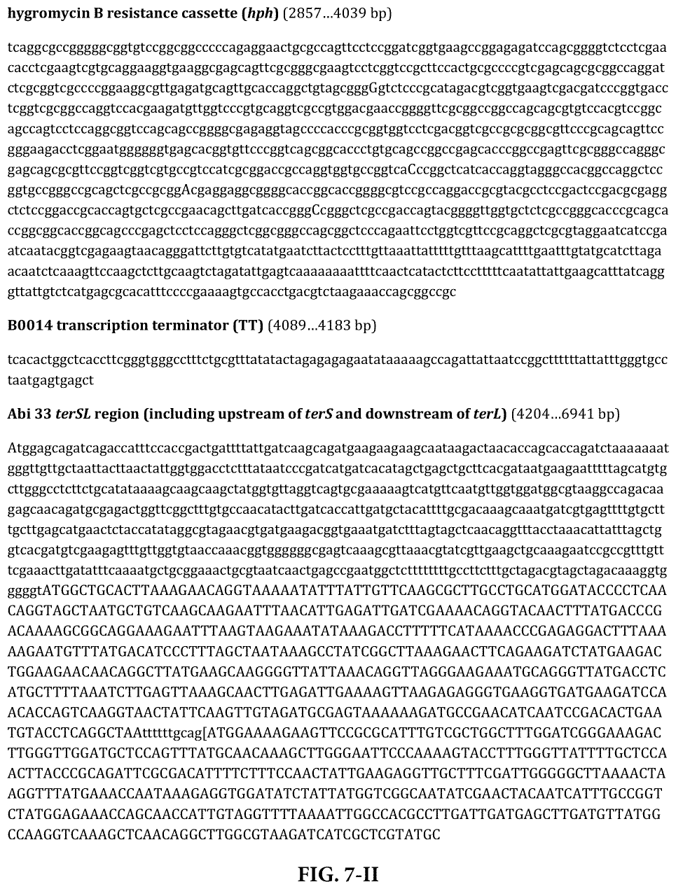

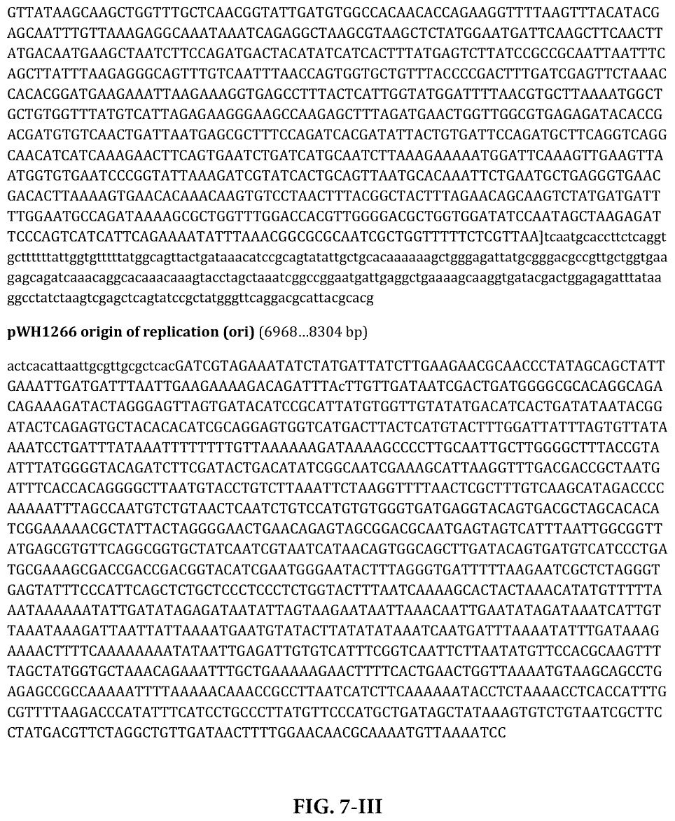

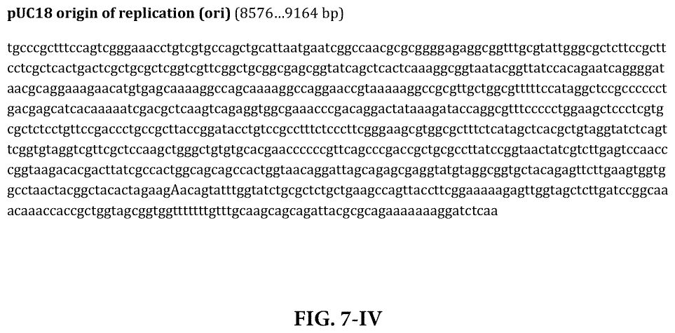

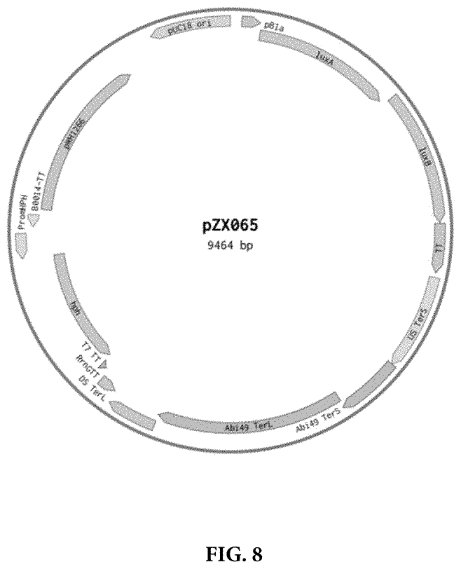

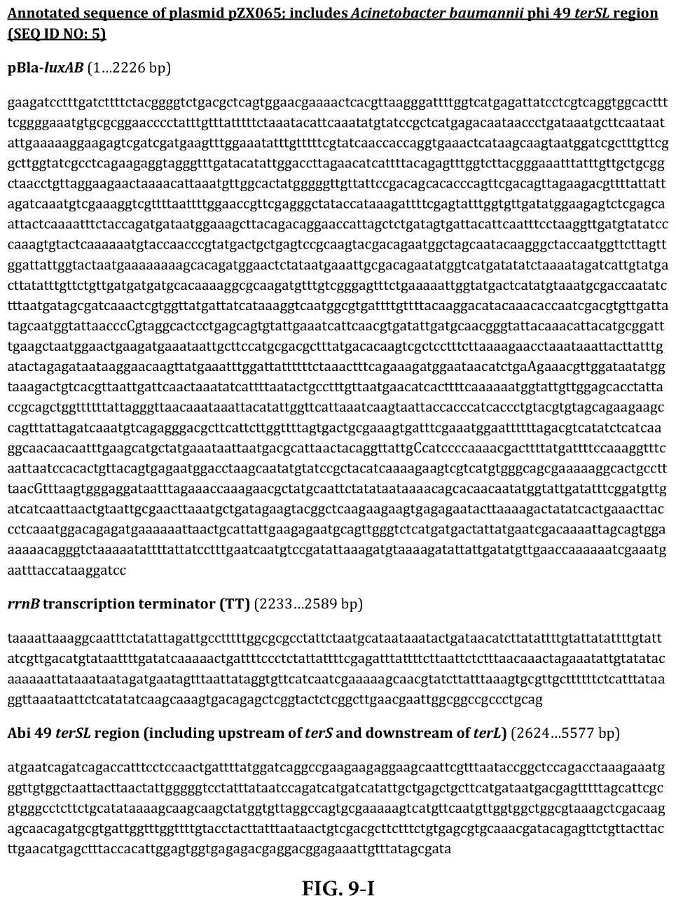

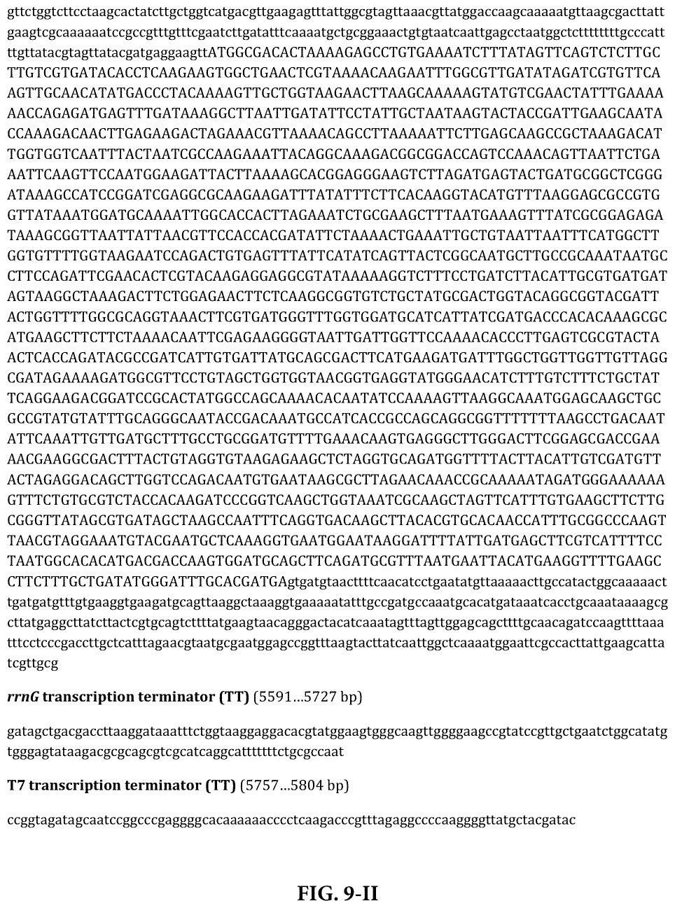

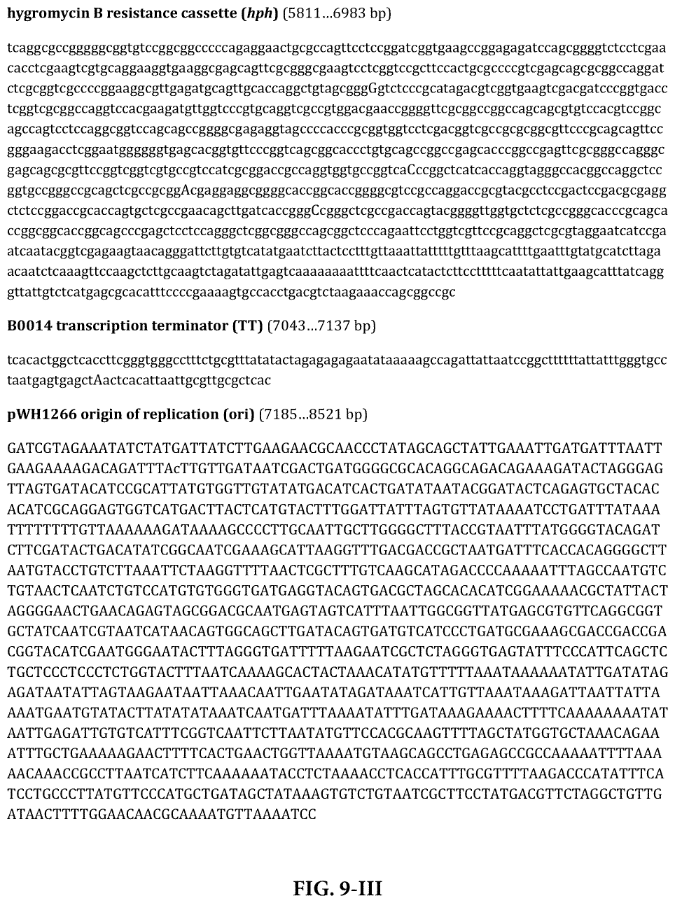

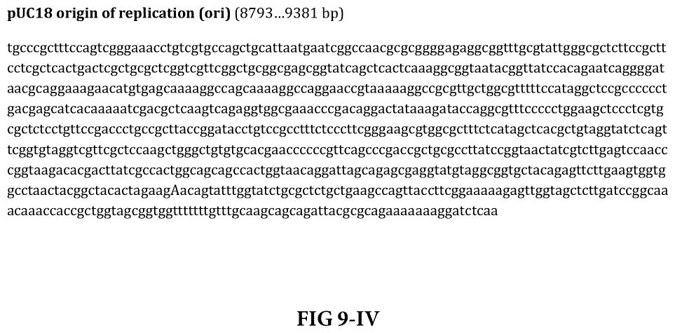

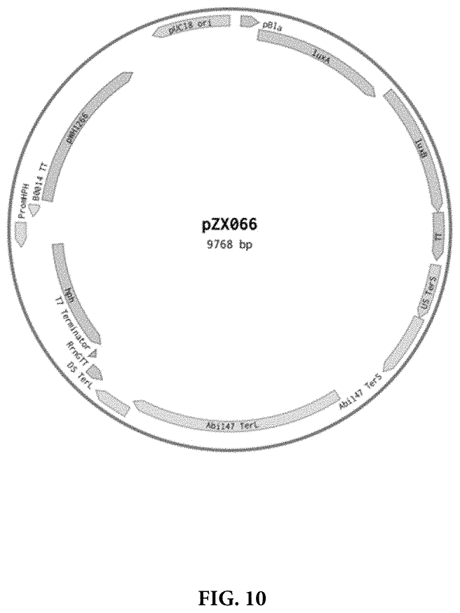

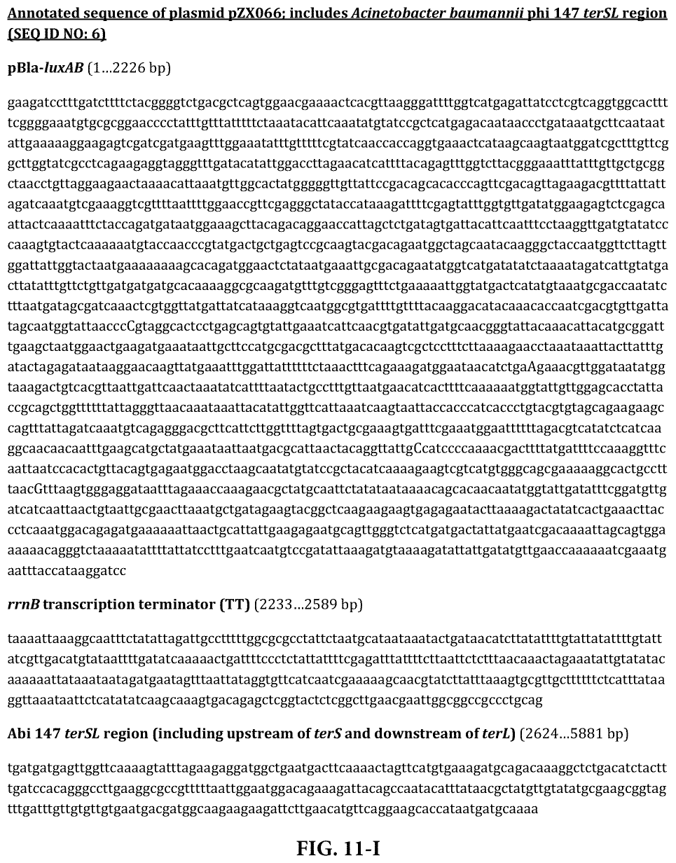

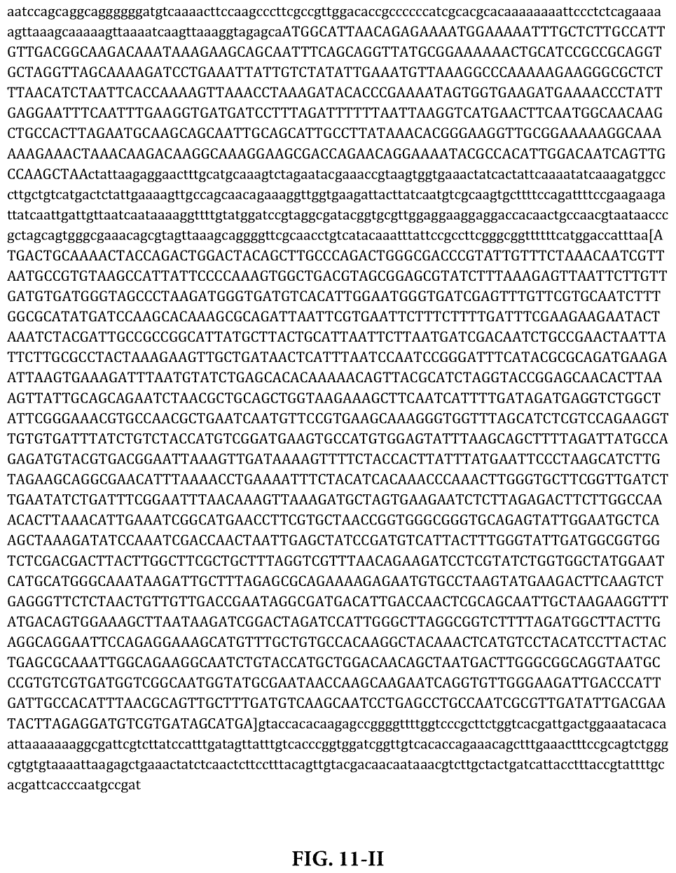

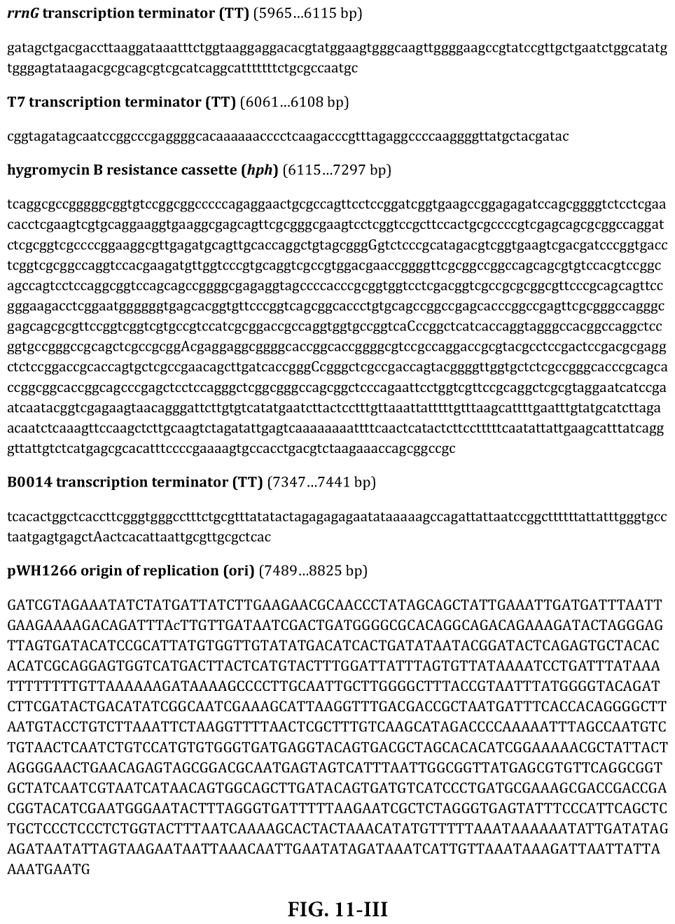

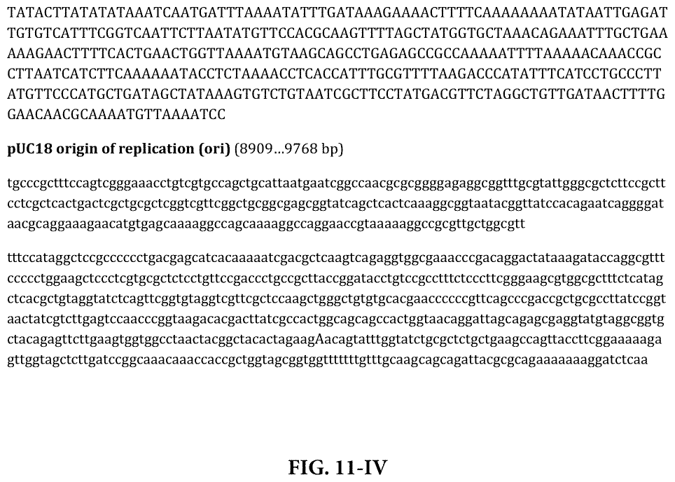

The Abi 33 packaging vector pZX058 (FIG. 6) was generated by cloning the full-length Abi 33 node3 terS and terL terminase genes with additional 700 bp upstream and 270 bp downstream sequences, respectively, into the cloning region 1. There were technical difficulties to clone Abi 49 and Abi 147 terminases into the cloning region 1, so pZX065 (FIG. 8) and pZX066 (FIG. 10) constructs were built by cloning the terminase into the cloning region 2. Specifically, pZX065 contained full-length Abi 49 terS and terL plus 700 bp upstream 366 bp downstream region sequence, respectively. The plasmid pZX066 contained full-length Abi 147 terS and terL plus the 414 bp upstream and 284 bp downstream sequences. The annotated nucleotide sequences of the plasmids pZX058, pZX065 and pZX066 are shown on FIGS. 7-I, -II, -III, -IV (SEQ ID NO: 4), 9-I, -II, -III, -IV (SEQ ID NO: 5) and 11-I, -II, -III, -IV (SEQ ID NO: 6), respectively.

New Abi 33, Abi 49, and Abi 147 non-replicative transduction particles using these stabilized packaging plasmids were generated as described in Example 1. Upon injection with the luminescence reaction substrate, 61%, 42%, and 41% of the strains were RLU-positive by Abi 33, Abi 49, and Abi 147 individual transduction particles, respectively. As a cocktail, there was an additive effect as 78% of the strains were RLU-positive (74 out of 95 A. baumannii strains). Furthermore, no cross-reactivity with non-A. baumannii strains were observed with the Abi 33, 49, and 147 Smarticles NRTPs used as a cocktail as measured by RLU. The results are shown on Table 4 where negative RLU indicate exclusivity of a total of 95 Enterobacteriaceae and Gram (−) strains tested. Abbreviations are as follows: Abi (Acinetobacter baumannii), Kpn (Klebsiella pneumoniae), Eco (Escherichia coli), Ecl (Enterobacter cloaceae), Pae (Pseudomonas aeruginosa), Kox (Klebsiella oxytoca), Eae (Enterobacter aerogenes), Cfi (Citrobacter freundii), Cko (Citrobacter koseri), Sms (Serratia marcescens).

TABLE 4

| | # of Strains | RLU +/−; |
|---|---|---|
| Species | Tested | (# of Positive) |
| Abi | 95 | + (74) |
| Kpn | 24 | − (0) |
| Eco | 24 | − (0) |
| Ecl | 23 | − (0) |
| Pae | 16 | − (0) |
| Kox | 2 | − (0) |
| Eae | 2 | − (0) |
| Cfi | 2 | − (0) |
| Cko | 1 | − (0) |
| Sms | 1 | − (0) |

### REFERENCES

1. Beggs, C B., G., K., M., A., & A., P. (2006). Acinetobacter spp. and the Clinical Environment. Indoor and Built Environment, 15(1), 19-24.
2. Peleg, A. Y., Harald Seifert, David L. Paterson. Acinetobacter baumannii: Emergence of a Successful Pathogen. Clinical Microbiology Reviews July 2008, 21 (3) 538-582; DOI: 10.1128/CMR.00058-07
3. Organization WHO. WHO Publishes List of Bacteria for which New Antibiotics are Urgently Needed.Geneva: WHO; 2017
4. Antunes L C, Visca P, Towner K J. Acinetobacter baumannii: evolution of a global pathogen. Pathog. Dis. 2014; 71:292-301. doi: 10.1111/2049-632X.12125.
5. Lemos, E V, de la Hoz F P, Einarson T R, McGhan W F, Quevedo E, Castañeda C, Kawai K. Carbapenem resistance and mortality in patients with Acinetobacter baumannii infection: systematic review and meta-analysis. Clin Microbiol Infect. 2014 May; 20(5):416-23
6. Li P, Niu W, Li H, Lei H, Liu W, Zhao X, Guo L, Zou D, Yuan X, Liu H, Yuan J and Bai C (2015) Rapid detection of Acinetobacter baumannii and molecular epidemiology of carbapenem-resistant A. baumannii in two comprehensive hospitals of Beijing, China. Front. Microbiol. 6:997. doi: 10.3389/fmicb.2015.00997
7. Martha R J Clokie, Andrew D Millard, Andrey V Letarov, Shaun Heaphy (2011) Phages in nature. Bacteriophage. 2011 January-February; 1(1): 31-45. doi: 10.4161/bact.1.1.14942
8. Chen, F, Kui Wang, Jeneen Stewart, Robert Belas. Induction of Multiple Prophages from a Marine Bacterium: a Genomic Approach. Appl Environ Microbiol. 2006 July; 72(7): 4995-5001.
9. Touchon, M, Cury J, Yoon E J, Krizova L, Cerqueira G C, Murphy C, Feldgarden M, Wortman J, Clermont D, Lambert T, Grillot-Courvalin C, Nemec A, Courvalin P, Rocha E P. The genomic diversification of the whole Acinetobacter genus: origins, mechanisms, and consequences. Genome Biol Evol. 2014 Oct. 13; 6(10):2866-82.
10. Sahl J. W, Del Franco M., Pournaras S., Colman R. E, Karah N., Dijkshoorn L., Zarrilli R. (2015). Phylogenetic and genomic diversity in isolates from the globally distributed Acinetobacter baumannii ST25 lineage Sci Rep 515188.10.1038/srep15188
11. Snitkin, Evan S., Adrian M. Zelazny, Clemente I. Montero, Frida Stock, LiliaMijares, NISC Comparative Sequence Program, Patrick R. Murray, Julie A. Segre. Genome-wide recombination drives diversification of epidemic strains of Acinetobacter baumannii. Proceedings of the National Academy of Sciences August 2011, 108 (33) 13758-13763
12. Chan, B. K., Abedon S T, Loc-Carrillo C. Phage cocktails and the future of phage therapy. Future Microbiol 2013; 8:769-83.
13. Aranda, J, Poza M, Pardo B G, Rumbo S, Rumbo C, Parreira J R, Rodriguez-Velo P, Bou G. A rapid and simple method for constructing stable mutants of Acinetobacter baumannii. BMC Microbiol. 2010 Nov. 9; 10:279.
14. Jacobs, A C, Thompson M G, Gebhardt M, Corey B W, Yildirim S, Shuman H A, Zurawski D V. Genetic Manipulation of Acinetobacter baumannii. Curr Protoc Microbiol. 2014 Nov. 3; 35:6G.2.1-11.

1. A bacterial cell packaging system for packaging a reporter plasmid comprising a reporter gene into a non-replicative transduction particle (NRTP) for introduction into an A. baumannii cell, the packaging system comprising:
a host A. baumannii cell;
a first nucleic acid construct inside the host A. baumannii cell comprising a bacteriophage genome having a disruption of one or more genes that encode packaging-related enzymatic activity, wherein the disruption prevents packaging of the bacteriophage genome into the NRTP, and wherein the bacteriophage genome is selected from the group consisting of the genome of bacteriophage Abi 33, the genome of bacteriophage Abi 49, and the genome of bacteriophage 147; and
a second nucleic acid construct inside the host A. baumannii cell and separate from the first nucleic acid construct, said second nucleic acid construct comprising a reporter nucleic acid molecule comprising a reporter gene and one or more genes that encode packaging-related enzymatic activity that complements the disruption on the bacteriophage genome and facilitates packaging of a replicon of the reporter nucleic acid molecule into the NRTP.

2. The bacterial cell packaging system of claim 1, wherein the one or more genes that encode packaging-related enzymatic activity comprises a terS gene, a terL gene or both terS and terL genes.

3. The bacterial cell packaging system of claim 1, wherein the disruption of the one or more genes that encode packaging-related enzymatic activity comprises a deletion of a nucleotide sequence selected from SEQ ID NO: 1, SEQ ID NO: 2 or SEQ ID NO: 3.

4. The bacterial cell packaging system of claim 1, wherein the reporter nucleic acid molecule comprises a nucleotide sequence selected from SEQ ID NO: 4, SEQ ID NO: 5 or SEQ ID NO: 6.

5. A method of producing non-replicative transduction particles (NRTPs) using the bacteria cell packaging system of claim 1, comprising the steps of:
a) inducing a lytic phase of the bacterial cell packaging system of claim 1, and
b) allowing the replicon of the reporter molecule to be packaged to produce the NRTPs.

6. The method of claim 5, wherein the disruption of the one or more genes that encode packaging-related enzymatic activity comprises a deletion of a nucleotide sequence selected from SEQ ID NO: 1, SEQ ID NO: 2 or SEQ ID NO: 3.

7. The method of claim 5, wherein the reporter nucleic acid molecule comprises a nucleotide sequence selected from SEQ ID NO: 4, SEQ ID NO: 5 or SEQ ID NO: 6.

8. A method of detecting A. baumannii in a sample comprising the steps of
a) providing NRTPs derived from bacteriophage Abi 33, NRTPs derived from bacteriophage Abi 49, NRTPs derived from bacteriophage Abi 147, or any combination of the above that are produced by the method of claim 5 to the sample;
b) providing conditions for the reporter gene to produce a detectable signal; and
c) detecting the presence or absence of the detectable signal to indicate the presence or absence of A. baumannii.

9. The method of claim 8 further comprising a step before or after providing NRTPs to the sample that comprises providing an antimicrobial agent to the sample and detecting for the presence or absence of the detectable signal to indicate whether the sample contains A. baumannii that is resistant or susceptible to the antimicrobial agent.

10. The method of claim 8, wherein the reporter nucleic acid molecule comprises a nucleotide sequence selected from SEQ ID NO: 4, SEQ ID NO: 5 or SEQ ID NO: 6.
